(19) United States
(12) Patent Application Publication
(10) Pub. No.: US 2024/0358801 A1
(43) Pub. Date: Oct. 31, 2024

(54) MODIFIED COLLOIDAL PARTICLES FOR USE IN THE TREATMENT OF HAEMOPHILIA A

(71) Applicant: Cantab Biopharmaceuticals Patents Limited, Valetta (MT)

(72) Inventor: RICHARD WOLF-GARRAWAY, London (GB)

(21) Appl. No.: 18/684,038

(22) PCT Filed: Aug. 17, 2022
(86) PCT No.: PCT/EP2022/073013

§ 371 Date: Feb. 15, 2024

(30) Foreign Application Priority Data
Aug. 17, 2021 (GB) 2111758.5

(51) Int. Cl. A61K 38/37; A61K 45/06; A61K 47/10; A61K 47/24; A61P 7/04

(52) U.S. Cl. CPC A61K 38/37; A61K 45/06; A61K 47/10; A61K 47/24; A61P 7/04

(57) ABSTRACT

The present invention relates to the use of colloidal particles for the treatment of haemophilia A in patients previously untreated or patients minimally treated with Factor VIII (FVIII). The invention also relates to compositions, methods, kits and dosage forms comprising colloidal particles for treating haemophilia A in patients previously untreated or patients minimally treated with Factor VIII (FVIII).

The present invention relates to the use of colloidal particles for the treatment of haemophilia A in patients previously untreated or patients minimally treated with Factor VIII (FVIII). The colloidal particle comprises a first and second amphipathic lipid wherein the second amphipathic lipid may be a phospholipid moiety derivatised with a biocompatible hydrophilic polymer such as polyethylene glycol (PEG). The invention also relates to compositions, methods, kits and dosage forms comprising colloidal particles.

The coagulation cascade that leads to blood coagulation is a multi-step process, involving many different proteins and factors, coupled with regulatory feedback mechanisms that enable the safe formation of a blood clot in the event of an injury. In disorders of the blood such as haemophilia, one or more of these factors may be defective or absent, leading to defective or poor quality clots.

In haemophilia A, the blood clotting Factor VIII (FVIII) is absent (severe haemophilia) or at low levels (moderate and mild haemophilia).

FVIII is a key protein in the ‘intrinsic’ pathway of the coagulation cascade, which when activated to FVIIIa, combines with FIXa to form the intrinsic ‘tenase’ complex that accelerates the conversion of FX to FXa, which participates in the conversion of prothrombin to thrombin which converts fibrinogen to fibrin, which forms the blood clot.

The conversion of FX to FXa can also be mediated by the ‘extrinsic’ initiation pathway. The formation of the extrinsic tenase complex of Tissue Factor (TF) and FVIIa (TF-FVIIa) initiates the clotting cascade, leading to the production of thrombin which also catalyses the activation of FVIII to FVIIIa. However, the extrinsic pathway is less efficient than the intrinsic pathway.

In the absence of FVIII, the coagulation cascade proceeds much more slowly since the cascade must rely on the extrinsic pathway alone to catalyse the conversion of FX to FXa.

In the absence of endogenous FVIII, it is common practice to administer replacement FVIII, either plasma-derived or recombinant, to the patient to restore their clotting capability. Patients may develop antibodies (inhibitors) to either exogenous FVIII (congenital haemophilia A—cHA) or to their own FVIII (acquired haemophilia A—aHA). The presence of inhibitors reduces the effectiveness of treating patients with exogenous FVIII as the protein is bound, neutralised and rapidly cleared from the circulation by the inhibitor antibody, making prophylactic treatment with replacement human FVIII very difficult or usually impossibly. A sub-optimal quantity of FVIII means that even if a clot can be formed at all, it is formed slowly or once formed is of a poor quality that is rapidly broken down.

There are currently two principal methods of dealing with the development of inhibitors: the use of inhibitor tolerance induction (ITI) or the use of bypass therapies. ITI utilises large, repeated doses of FVIII over several months to induce tolerance in the immune system to FVIII, with the aim of enabling the patient to return to a normal dosing regimen. The therapy is not always effective and the repeated high-dose injections of FVIII over several months are both unpleasant for the patient and extremely costly, representing a significant healthcare system cost.

Bypass therapies avoid the problem of inhibitors by ‘bypassing’ the amplification phase entirely. These therapies might simply boost the extrinsic pathway by supplying additional FVIIa (e.g. NovoSeven) or may supply a mixture of active and inactivated factors that boost the cascade in the absence of FVIII, for example FEIBA (a mixture of FVIIa, FIX, FIXa, FX, FXa, prothrombin and thrombin). More recent developments have attempted to replace the role of FVIIIa in bringing FIXa and FX together through the use of a bi-specific antibody.

The use of replacement FVIII to treat either congenital haemophilia A or acquired haemophilia A is well established in clinical practice. However, the efficacy of such products is limited due to the occurrence of inhibitory antibodies being present in some patients that reduces the effectiveness of exogenously delivered Factor VIII. Inhibitors arise in 25-35% of congenital haemophilia A sufferers (‘inhibitor patients’) as a response to the exogenous FVIII they receive; in acquired haemophilia A the disease arises as the patient develops inhibitors to their own FVIII due to autoimmunity.

The wild-type FVIII molecule comprises 2332 amino acids, organised in 6 domains: A1-A2-B-A3-C1-C2. Together the A1-A2-B domains comprise the ‘Heavy Chain’ (HC) and the A3-C1-C2 domains comprise the ‘Light Chain’ (LC) and these chains are linked non-covalently. In life, FVIII will normally associate with von Willebrand's Factor (VWF) in circulation. VWF facilitates the transport of FVIII and protects it from premature inactivation and clearance (Mannucci, P. M. et al. (2014) Novel investigations on the protective role of the FVIII/VWF complex in inhibitor development. Haemophilia. 20(suppl. 6), 2-16.). The association with VWF is also associated with reduced immunogenicity, efficacy in the presence of inhibitors and utility in immunotolerance treatment. The use of commercial concentrates of plasma-derived FVIII (pdFVIII) containing VWF were shown to be less immunogenic than recombinant concentrates of FVIII (rFVIII) in the SIPPET trial (Peyvandi, F. et al. (2016) A randomized trial of Factor VIII and neutralizing antibodies in Hemophilia A, N Engl J Med. 374, 2054-64). The reduced immunogenicity of the pdFVIII concentrates is associated with the VWF chaperone, which it Is thought either masks critical epitopes on the FVIII molecule, and/or prevents its endocytosis by dendritic cells (Astermark, J. (2015) FVIII inhibitors: pathogenesis and avoidance. Blood. 125(13), 2045-51). Recombinant FVIII molecules also have the additional complication that most of these molecules are not humanised but are produced in non-human cell lines, resulting in the presence of non-human glycan epitopes potentially enhancing their immunogenicity.

The development of inhibitors to these epitopes in FVIII remains the most frequent side effect of haemophilia treatment (Van den Berg et al. (2020). ITI treatment is not a first-choice in children with hemophilia A and low-responding inhibitors: Evidence from a PedNet study. Coagulation and Fibrinolysis. 120, 1166-1172). The risk is high in previously untreated patients (PUPs) with an overall incidence of up to 40% (ibid.), inactivating FVIII activity and requiring alternative and costly measures to protect these patients. In some patients, the use of immune tolerance induction therapy, a long and costly technique, can eradicate inhibitors but the technique does not work for all patients, preventing them from using replacement FVIII therapy. The risk of inhibitor development is highest during the first 20 exposure days (EDs) to replacement FVIII and persists up to 75 EDs (Liesner, R. J. et al. (2021) Simoctocog alfa (Nuwiq™) in previously untreated patients with severe haemophilia A: final results of the NuProtect study. Thromb Haemost. online). Patients must be monitored for the development of inhibitors over this period.

Clearly it would preferable if these previously untreated patients could be treated with a FVIII therapy that is unlikely to be immunogenic from the start, to reduce the chance of ever developing inhibitors.

There is therefore a need for a non-immunogenic FVIII replacement therapy that reduces the chance of ever developing inhibitors.

The present invention therefore provides PEGylated colloidal particles for use in the treatment of haemophilia A to lower the risk of the development of inhibitors in previously untreated and minimally treated patients when treated with FVIII, by a combination of epitope shielding and half-life extension.

It has been discovered that PEGylated liposomes appear to prevent the generation of anti-FVIII antibodies in inhibitor-prone models, as well as enabling the dose/dosing interval to be reduced.

This provides a safer method by which to treat previously untreated patients (PUPs) and minimally treated patients (MTPs—less than 50 exposure days (EDs) to a Factor VIII (FVIII) therapy).

### SUMMARY OF THE INVENTION

The present invention provides compositions, methods, kits and dosage forms comprising a colloidal particle for treating haemophiliac patients with a deficiency in FVIII, and who do not have inhibitors to FVIII. The compositions, methods, kits and dosage forms may further optionally comprise a non-ionic surfactant and/or FVIII.

In a first aspect of the invention there is provided a composition comprising a colloidal particle comprising (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI), wherein said second amphipathic lipid comprises a phospholipid moiety derivatised with a biocompatible hydrophilic polymer, for use in the treatment of haemophilia A in a subject. The subject has received less than 50 exposure days to a Factor VIII (FVIII) therapy.

The biocompatible hydrophilic polymer may be selected from the group consisting of polyalkylethers, polylactic acids and polyglycolic acids, preferably, the biocompatible hydrophilic polymer is polyethylene glycol (PEG). The polyethylene glycol may have a molecular weight of between about 500 to about 5000 Daltons, preferably about 2000 Daltons or about 5000 Daltons.

The second amphipathic lipid may be N-(Carbonyl-methoxypolyethyleneglycol)-1,2-distearoyl-sn-glycero-3-phosphoethanolamine (DSPE-PEG) such as N-(Carbonyl-methoxypolyethyleneglycol-2000)-1,2-distearoyl-sn-glycero-3-phosphoethanolamine (DSPE-PEG2000) or N-(Carbonyl-methoxypolyethyleneglycol-5000)-1,2-distearoyl-sn-glycero-3-phosphoethanolamine (DSPE-PEG5000).

The phosphatidyl choline (PC) may be 1-palmitoyl-2-oleoyl-sn-glycero-3-phosphocholine (POPC).

The first amphipathic lipid and the second amphipathic lipid may be in a molar ratio of from 90 to 110:10 to 1 or 90 to 99:10 to 1, such as 100:3 or 97:3.

The colloidal particle may further comprise (iii) a non-ionic surfactant. The non-ionic surfactant may be selected from the group consisting of a polyoxyethylene sorbitan, a polyhydroxyethylene stearate and a polyhydroxyethylene laurylether. The non-ionic surfactant may be polyoxyethylene (20) sorbitan monooleate. The colloidal particle may compromise the first amphipathic lipid and the second amphipathic lipid to the non-ionic surfactant in a ratio of from 30:1 to 2:1 w/w ({first amphipathic lipid+second amphipathic lipid}:{non-ionic surfactant}).

The first amphipathic lipid to the second amphipathic lipid to the non-ionic surfactant may be in a ratio of from 10 to 40:1:0 to 4 w/w ({first amphipathic lipid}:{second amphipathic lipid}:{non-ionic surfactant}).

The composition may further comprise a Factor VIII (FVIII) molecule. The colloidal particle and the Factor VIII (FVIII) molecule may be in a stoichiometric ratio of from 1 to 90:1 such as 10 to 20:1 or 5 to 10:1.

The haemophilia A may be congenital haemophilia A (cHA) or acquired haemophilia A (aHA).

The composition may further comprise a therapeutically active compound. The composition may also further comprise an excipient, diluent and/or adjuvant.

The subject may be a paediatric patient. The subject may also not have received FVIII therapy.

The compositions of the invention may be formulated in an aqueous suspension ready for use, or the compositions may be prepared as a lyophilised formulation. Lyophilised formulations of the invention may be supplied as separate dosage forms along with a suitable diluent, adjuvant or excipient provided also, e.g. a physiologically acceptable buffer. As described herein, such compositions may additionally comprise Factor VIII as a separate dosage form, or formulated with the colloidal particles as described herein.

In a second aspect of the invention there is provided a method of treating haemophilia A in a subject comprising the step of administering a composition comprising a colloidal particle. The colloidal particle comprises (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI). The second amphipathic lipid comprises a phospholipid moiety derivatised with a biocompatible hydrophilic polymer. The subject has received less than 50 exposure days to a Factor VIII (FVIII) therapy.

The biocompatible hydrophilic polymer may be selected from the group consisting of polyalkylethers, polylactic acids and polyglycolic acids, preferably, the biocompatible hydrophilic polymer is polyethylene glycol (PEG). The polyethylene glycol may have a molecular weight of between about 500 to about 5000 Daltons, preferably about 2000 Daltons or about 5000 Daltons.

The second amphipathic lipid may be N-(Carbonyl-methoxypolyethyleneglycol)-1,2-distearoyl-sn-glycero-3-phosphoethanolamine (DSPE-PEG) such as N-(Carbonyl-methoxypolyethyleneglycol-2000)-1,2-distearoyl-sn-glycero-3-phosphoethanolamine (DSPE-PEG2000) or N-(Carbonyl-methoxypolyethyleneglycol-5000)-1,2-distearoyl-sn-glycero-3-phosphoethanolamine (DSPE-PEG5000).

The phosphatidyl choline (PC) may be 1-palmitoyl-2-oleoyl-sn-glycero-3-phosphocholine (POPC).

The colloidal particle may further comprise (iii) a non-ionic surfactant.

The composition of the method may further comprise a Factor VIII (FVIII) molecule. Alternatively, the method may comprise a further step of separately or subsequently administering a composition comprising a Factor VIII (FVIII) molecule.

The haemophilia A may be congenital haemophilia A (cHA) or acquired haemophilia A (aHA).

The subject may be a paediatric patient. The subject may also not have received a FVIII therapy.

In a third aspect of the invention there is provided a kit comprising (i) a composition comprising a colloidal particle and (ii) a composition comprising a Factor VIII (FVIII) molecule for use in the treatment of haemophilia A in a subject. The subject has received less than 50 exposure days to a Factor VIII (FVIII) therapy. The colloidal particle comprises (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI). The second amphipathic lipid comprises a phospholipid moiety derivatised with a biocompatible hydrophilic polymer.

The colloidal particle may further comprise (iii) a non-ionic surfactant.

In a fourth aspect of the invention there is provided a kit comprising (i) a composition comprising a colloidal particle and (ii) a composition comprising a Factor VIII (FVIII) molecule for separate, simultaneous or subsequent use in the treatment of haemophilia A in a subject. The subject has received less than 50 exposure days to a Factor VIII (FVIII) therapy. The colloidal particle comprises (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI). The second amphipathic lipid comprises a phospholipid moiety derivatised with a biocompatible hydrophilic polymer.

The colloidal particle may further comprise (iii) a non-ionic surfactant.

In a fifth aspect of the invention there is provided a dosage form of a pharmaceutical composition comprising a colloidal particle comprising (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI), wherein said second amphipathic lipid comprises a phospholipid moiety derivatised with a biocompatible hydrophilic polymer for use in the treatment of haemophilia A in a subject. The subject has received less than 50 exposure days to a Factor VIII (FVIII) therapy.

The colloidal particle may further comprise (iii) a non-ionic surfactant.

The invention is suited to haemophiliac patients with a deficiency in FVIII, and who do not have inhibitors to FVIII.

In an embodiment, the method is for treating previously untreated patients (PUPs) or minimally treated patients (MTPs) with mild, moderate or severe haemophilia A, to minimise the chance of previously untreated patients developing inhibitors.

In another embodiment, the method is for treating patients who have previously been shown to be inhibitor prone but are currently inhibitor-free, to prevent the recurrence of inhibitors.

In an embodiment, the method is for treating patients who had previously developed inhibitors, but following a course of immune tolerance induction therapy are now inhibitor-free, to prevent recurrence of inhibitors.

In another embodiment, the method is for treating patients who are undergoing immune tolerance induction therapy to prevent further stimulation of the immune system.

In an embodiment, the method is for treating mild, moderate or severe haemophilia A patients who have not yet developed inhibitors and who wish to lower their risk of developing inhibitors to their FVIII replacement therapy.

### DESCRIPTION OF THE INVENTION

The invention provides compositions, methods, kits and dosage forms comprising a colloidal particle for use in the treatment of haemophilia A in a subject wherein the subject has received less than 50 exposure days to a Factor VIII (FVIII) therapy.

A Factor VIII (FVIII) therapy may be a Factor VIII (FVIII) replacement therapy such as plasma-derived or recombinant FVIII, to a patient/subject to restore their clotting capability. In the cases where patients/subjects have developed inhibitors, the patient/subject may receive a therapy such as ‘bypassing’ therapy or inhibitor tolerance induction (ITI) therapy. Bypass therapies include FVIIa (e.g. NovoSeven) or a mixture of active and inactivated factors, for example FEIBA (a mixture of FVIIa, FIX, FIXa, FX, FXa, prothrombin and thrombin). Inhibitor tolerance induction (ITI) utilises large, repeated doses of FVIII over several months to induce tolerance in the immune system to FVIII.

Inhibitors or antibody inhibitors to FVIII refer to antibodies, also interchangeably known antibody inhibitors or neutralising antibodies, to FVIII. The antibodies may be auto-antibodies to endogenous FVIII or antibodies to exogenous FVIII.

In accordance with the first aspect described above, the colloidal particle comprises (i) a first amphipathic lipid and (ii) a second amphipathic lipid. The first amphipathic lipid is a phosphatidylcholine (PC) moiety. A suitable example of a phosphatidyl choline (PC) moiety may be 1-palmitoyl-2-oleoyl-sn-glycero-3-phosphocholine (POPC). The second amphipathic lipid is a phospholipid moiety selected from the group consisting of phosphatidylethanolamine (PE), phosphatidyl serine (PS), phosphatidyl inositol (PI). A suitable example of phosphatidyl ethanolamine (PE) may be 1,2-distearoyl-sn-glycero-3-phosphoethanol-35 amine (DSPE). Aminopropanediol distearoyl (DS) lipid is a carbamate-linked uncharged lipopolymer which is also an amphipathic lipid. Other examples of phosphatidyl ethanolamine (PE) include DPPE, DMPE and DOPE.

The colloidal particles of the invention are typically in the form of lipid vesicles or liposomes and are well known in the art. References to colloidal particles in the present specification include liposomes and lipid vesicles unless the context specifies otherwise.

The second amphipathic lipid is a phospholipid moiety derivatised with a biocompatible hydrophilic polymer.

The purpose of the biocompatible hydrophilic polymer is to sterically stabilize the colloidal particle, thus preventing fusion of the colloidal particle in vitro, and allowing the colloidal particle to escape adsorption by the reticuloendothelial system in vivo. The biocompatible hydrophilic polymer may be selected from the group consisting of polyalkylethers, polylactic acids and polyglycolic acids. The biocompatible hydrophilic polymer may be polyethyleneglycol (PEG). The polyethylene glycol may be branched or unbranched. The biocompatible polymer may have a molecular weight of between about 100 to about 10,000 Da, suitably of from about 2000 to about 5000 Da, with preferred values of about 100 Da, 250 Da, 350 Da, 550 Da, 750 Da, 1000 Da, 1500 Da, 2000 Da, 2500 Da, 3000 Da, 3500 Da, 4000 Da, 4500 Da, 5000 Da, 5500 Da, 6000 Da, 6500 Da, 7000 Da, 7500 Da, 8000 Da, 8500 Da, 9500 Da and 10,000 Da. A suitable example of a phospholipid derivatised with a biocompatible hydrophilic polymer may be N-(Carbonyl-methoxypolyethyleneglycol)-1,2-distearoyl-sn-glycero-3-phosphoethanolamine (DSPE-PEG) such as N-(Carbonyl-methoxypolyethyleneglycol-2000)-1,2-distearoyl-sn-glycero-3-phosphoethanolamine (DSPE-PEG(2000)) and N-(Carbonyl-methoxypolyethyleneglycol-5000)-1,2-distearoyl-sn-glycero-3-phosphoethanolamine (DSPE-PEG5000).

The first amphipathic lipid and the second amphipathic lipid may be provided in a molar ratio of from 90 to 110:10 to 1, 90 to 100:10 to 1, 90 to 99:10 to 1, 93 to 99:7 to 1, 95 to 99:5 to 1 suitably 100:3, 100:3, 99:3, 98:3, 97:3, 96:3 or 95:3. The molar ratio of 97.3 may also be expressed as a molar ratio of 32.4:1, likewise the molar ratio of 100:3 may be expressed as a molar ratio of 33.2:1. The ratio of the first amphipathic lipid and the second amphipathic lipid may also be expressed as a weight/weight ratio for example, 1:1 to 20:1 w/w, suitably 2:1 to 12:1 w/w or 4:1 to 9:1 w/w, for example 4:1, 5:1, 6:1, 9:1 or 12:1 w/w. Suitably, the composition may comprise a colloidal particle composed of a mixture of palmitoyl-oleoyl phosphatidyl choline (POPC) and 1,2-distearoyl-sn-glycero-3-phosphoethanol-amine (DSPE) in a molar ratio (POPC:DSPE) of from 90 to 99:10 to 1, 93 to 99:7 to 1, 95 to 99:5 to 1 suitably 97:3. Expressed as a weight/weight ratio this may be, for example, 1:1 to 20:1 w/w, suitably 2:1 to 12:1 w/w, for example 4:1, 5:1, 6:1, 9:1 or 12:1 w/w.

In one instance, the colloidal particle may be composed of 1-palmitoyl-2-oleoyl-sn-glycero-3-phosphocholine (POPC) and N-(Carbonyl-methoxypolyethyleneglycol-2000)-1,2-distearoyl-sn-glycero-3-phosphoethanolamine (DSPE-PEG(2000)) in a 97:3 molar ratio or 9:1 w/w ratio.

The colloidal particle may have a mean particle size (average particle size) ranging from 0.05 to 0.0.3 μm diameter, suitably around 0.1, 0.15, 0.2 or 0.25 microns (μm). The average particle size (mean particle size) may be from 100 to 130 nanometres (nm), suitably 110 to 120 nm, 112 to 118 nm, 150 to 170 nm, 155 to 165 nm, more suitably 110, 112, 114, 116, 118, 120, 160, 162, 164, 166, 168 or 170 nm.

Mean particle size may be measured using a Malvern Zetasizer Ultra ZSU 5700. This instrument determines particle size by light scattering, whereby the back-scatter from laser light shone into the sample and hitting particles is detected at an angle of 1730 (1730 being almost back on itself & hence the term back-scatter). Brownian motion of particles causes the light to be scattered at different intensities. Because the velocity of Brownian motion relates to particle size, particle size can be inferred via the Stokes-Einstein relationship.

Mean particle size corresponds to the mean diameter of the colloidal particle. The polydispersity index (PDI) quoted in relation to particle size measurements corresponds to the measure of distribution around the mean diameter of the colloidal particle. For example, in the present invention, the polydispersity index may be a maximum of 0.2, suitably 0.15, 0.12, 0.1 or 0.5.

The PDI is calculated as the square of the standard deviation/mean, i.e. PDI=(s/m)².

From the mean particle size and the polydispersity index, the standard deviation can be calculated. Twice the standard deviation facilitates the calculation of the 95% confidence intervals around the particle size, i.e. the range in which 95% of the colloidal particles in the sample lie.

Suitably, the 95% mean particle size may be 50 to 500 nm, suitably 50 to 290 nm, 50 to 285 nm, 65 to 265 nm, 65 to 260 nm, 65 to 180 nm, 65 to 175 nm, 65 to 170 nm, 65 to 165 nm, 65 to 160 nm, more suitably 65 to 173 nm, 64 to 161 nm, 65 to 263 nm or 54 to 282 nm.

The colloidal particle may further comprise (iii) a surfactant such as a non-ionic surfactant. The non-ionic surfactant may be selected from the group consisting of a polyoxyethylene sorbitan, a polyhydroxyethylene stearate and a polyhydroxyethylene laurylether. The non-ionic surfactant may be polyoxyethylene (20) sorbitan monooleate (also known as polysorbate 80 or Tween 80). The first amphipathic lipid and the second amphipathic lipid to the non-ionic surfactant may be provided in a ratio of from 30:1 to 2:1 w/w, suitably 25:1, 20:1, 16:1, 15:1, 14:1, 13:1, 12:1, 11:1, 10:1, 9:1, 8:1. or 5:1 w/w ({first amphipathic lipid+second amphipathic lipid}:{non-ionic surfactant}). Expressed as a molar ratio, this may be for example 10 to 20:1, 12 to 18:1, 14 to 16:1, suitably 14:1, 15:1 or 16:1. The surfactant concentration may be from 0.25% to 5% by weight, for example 1% to 3%, 1 to 2%, some exemplary values may be 0.47%, 0.85%, or 3.5%.

The non-ionic surfactant may also be PEGylated. A PEGylated non-ionic surfactant may be polyoxyethylene (20) sorbitan monooleate (also known as polysorbate 80 or Tween 80). The polyethylene glycol may be branched or unbranched. The biocompatible polymer may have a molecular weight of between about 100 to about 10,000 Da, suitably of from about 2000 to about 5000 Da, with preferred values of about 100 Da, 250 Da, 350 Da, 550 Da, 750 Da, 1000 Da, 1500 Da, 2000 Da, 2500 Da, 3000 Da, 3500 Da, 4000 Da, 4500 Da, 5000 Da, 5500 Da, 6000 Da, 6500 Da, 7000 Da, 7500 Da, 8000 Da, 8500 Da, 9500 Da and 10,000 Da.

The non-ionic surfactant may be associated with the colloidal particle, incorporated into the lipid bilayer membrane of the colloidal particle, incorporated into the outer layer of the lipid bilayer membrane of the colloidal particle or incorporated into the inner layer lipid bilayer membrane of the colloidal particle.

Accordingly, the first amphipathic lipid to the second amphipathic lipid to the non-ionic surfactant may be provided in a ratio of from 2 to 10:1:0 to 2, 3 to 9:1:0.5 to 1.5, 4 to 9:1:0.5 to 1 w/w, suitably 9:1:0, 9:1:1, 4:1:0 or 4:1:0.5 w/w ({first amphipathic lipid}:{second amphipathic lipid}:{non-ionic surfactant}).

Expressed as a molar ratio this may be, for example, 30 to 40:1:0 to 5, 30 to 35:1:0 to 2.5, 30 to 35:1:0 to 2.5, suitably 33:1:0, 33:1:2 or 32:1:3.

The composition may further comprise a Factor VIII (FVIII) molecule or a fragment thereof. Where the composition comprises a fragment of Factor VIII, the Factor VIII fragment may suitably be an active fragment in which the fragment retains the biological activity, or substantially the same biological activity as the native Factor VIII molecule. For example, one such active fragment is the B-domain truncated Factor VIII. It is further possible that the composition may comprise both the native blood factor and a fragment thereof. The colloidal particle and the Factor VIII (FVIII) molecule may be provided in a stoichiometric ratio of from 1 to 90:1, suitably 2 to 90:1, 5 to 85:1, 6 to 10:1, 7 to 8:1, 7.5 to 20:1, 10 to 80:1, 10 to 15:1, 10 to 16:1, 10 to 20:1, 13 to 19:1, 15 to 16:1, 15 to 75:1, 20 to 70:1, 25 to 65:1, 30 to 60:1, 35 to 55:1, 40 to 50:1, such as 10 to 20:1 and 5 to 10:1. Alternatively expressed, the colloidal particle and the Factor VIII (FVIII) molecule may be provided in a stoichiometric ratio of 1:1, 2:1, 5:1, 7.5:1, 10:1, 15:1, 16:1, 17:1, 18:1, 19:1, 20:1, 22:1, 25:1, 26:1, 27:1, 28:1, 29:1, 30:1, 35:1, 40:1, 45:1, 50:1, 55:1, 60:1, 65:1, 70:1, 75:1, 80:1, 85:1, 86:1, 90:1 such as 15.5:1, 13:1, 8:1, 7.7:1, 7:1. More specifically for full-length FVIII molecules the stoichiometric ratio may be 10:1 to 19:1 and optimally 10 to 15:1 or 5 to 10:1 and optimally 7.5:1. For beta-domain deleted or beta-domain truncated FVIII molecules the ranges may be 13 to 19:1 and optimally 10 to 16:1 or 6 to 10:1 and optimally 8:1.

Without wishing to be bound by theory, there is a presumption that the excess colloidal particles present in the composition in an amount sufficient to allow free colloidal particles to reversibly bind other core blood factors (for example FVII and FIX in the case of haemophilia A) which with an amount of particle-associated Factor VIII (FVIII) may be captured and reversibly bound to the platelets following administration, to concentrate factors at the platelet and boost the extrinsic blood coagulation pathway.

Factor VIII may be from any suitable source and may be a recombinant protein produced by recombinant DNA technology using molecular biological techniques or synthesised chemically or produced transgenically in the milk of a mammal, or the Factor VIII may be isolated from natural sources (e.g. purified from blood plasma). Suitably the Factor VIII is a mammalian Factor VIII, such as a human Factor VIII.

Blood factors, such as Factor VIII, are characterised by the property of surface adhesion. This is a necessary feature of the coagulation cascade which requires that enzymes and cofactors adhere to other participants in the cascade, to the surface of platelets and to tissue at the site of injury. It is particularly important that a blood clot remains at the site of injury and does not drift to cause a dangerous thrombosis. This property presents a challenge in the formulation of drug products, since blood factors such as Factor VIII will adhere excessively to any glass and plastic surfaces. In practical terms this is mitigated by the extensive use of a non-ionic surfactant such as polyoxyethylene (20) sorbitan monooleate polysorbate (Tween® 80).

To determine the stoichiometric ratio of the colloidal particle to the Factor VIII (FVIII) molecule, the following calculation should be performed. First the molecules of FVIII per IU of FVIII should be determined. Note, the mass of FVIII varies depending on the variant of FVIII (for example full length versus β-domain deleted). Second, the number of particles per gram of colloidal particle should be determined. Finally, a stoichiometric ratio can be determined accordingly. Example calculations with 35 IU/kg FVIII (both beta-domain deleted and full-length) and 22 mg/kg PEGLip are as follows:

### Calculation Example 1, a Beta-Domain Deleted rFVIII

| FVIII, e.g. Nuwiq | | | | |
|---|---|---|---|---|
| Specific activity | 9,500 | | IU/mg | A |
| Mass of 1 IU of FVIII | 0.105 | | mg | B = 1/A × 1,000 |
| = | 1.05 × 10⁻⁷ | | g | C = B/1,000,000 |
| Molecular mass of FVIII | 170,000 | | g/mol | D |
| Moles in 1 IU | 6.19 × 10⁻¹³ | | Moles | E = C/D |
| N(A) | 6.02 × 10²³ | | Molecules/mole | F |
| Molecules of FVIII per IU | 3.73 × 10¹¹ | | Molecules/IU | G = E × F |
| PEGLip | POPC | DSPE | | |
| Mass of 1 g PEGLip solids | 0.9 | 0.1 | g | H |
| Molecular mass | 760.1 | 2748 | g/mol | I |
| Moles/g PEGLip solids | 1.19 × 10⁻³ | 3.64 × 10⁻⁵ | Moles | J = H/I |
| | J1 | J2 | | |
| Total moles lipid | 1.22 × 10⁻³ | | Moles | K = J1 + J2 |
| N(A) | 6.02 × 10²³ | | Molecules/mole | L |
| N(tot) | 80,047 | | Lipid/surface | M |
| N(Lipo) | 9.18 × 10¹⁵ | | Liposomes/g | N = K/L × M |
| | | | PEGLip solids | |
| Ratio Calculation | | | | |

| | 1.30 × 10¹³ | Molecules | O = G × 35 |
|---|---|---|---|
| 35 IU/kg BDDFVIII | | | |
| 22 mg/kg PEGLip | 2.02 × 10¹⁴ | Liposomes | P = N × 22 |
| Ratio | 15:1 | Liposomes:FVIII | Q = P/O |
| | | (rounded) | |

### Calculation Example 2, a Full-Length rFVIII

| FVIII, e.g. Kogenate | | | | |
|---|---|---|---|---|
| Specific activity | 4,000 | | IU/mg | A |
| Mass of 1 IU of FVIII | 0.250 | | mg | B = 1/A × 1,000 |
| = | 2.50 × 10⁻⁷ | | g | C = B/1,000,000 |
| Molecular mass of FVIII | 265,000 | | g/mol | D |
| Moles in 1 IU | 9.43 × 10⁻¹³ | | Moles | E = C/D |
| N(A) | 6.02 × 10²³ | | Molecules/mole | F |
| Molecules of FVIII per IU | 5.68 × 10¹¹ | | Molecules/IU | G = E × F |
| PEGLip | POPC | DSPE | | |
| Mass of 1 g PEGLip solids | 0.9 | 0.1 | g | H |
| Molecular mass | 760.1 | 2748 | g/mol | I |
| Moles/g PEGLip solids | 1.19 × 10⁻³ | 3.64 × 10⁻⁵ | Moles | J = H/I |
| | J1 | J2 | | |
| Total moles lipid | 1.22 × 10⁻³ | | Moles | K = J1 + J2 |
| N(A) | 6.02 × 10²³ | | Molecules/mole | L |
| N(tot) | 80,047 | | Lipid/surface | M |
| N(Lipo) | 9.18 × 10¹⁵ | | Liposomes/g | N = K/L × M |
| | | | PEGLip solids | |
| Ratio Calculation | | | | |

| | | | |
|---|---|---|---|
| 35 IU/kg BDDFVIII | 1.99 × 10¹³ | Molecules | O = G × 35 |
| 22 mg/kg PEGLip | 2.02 × 10¹⁴ | Liposomes | P = N × 22 |
| Ratio | 10:1 | Liposomes:FVIII | Q = P/O |
| | | (rounded) | |

The composition may comprise a further therapeutically active compound or molecule, e.g. an anti-inflammatory drug, analgesic or antibiotic, or other pharmaceutically active agent which may promote or enhance the activity of Factor VIII.

The composition may further comprise any suitable excipient, diluent and/or adjuvant. Suitable diluents, such as buffers may be formulated with a water-soluble salt of an alkali metal or an alkaline earth metal and a suitable acid. Suitable buffer solutions may include, but are not limited to amino acids (for example histidine), salts of inorganic acids (for example an acid selected from the group consisting of citric acid, lactic acid, succinic acid, citric acid and phosphoric acid) and alkali metals or alkaline earth metals, (for example sodium salts, magnesium salts, potassium salts, lithium salts or calcium salts—exemplified as sodium chloride, sodium phosphate or sodium citrate). Examples of such excipient, buffer and/or adjuvants, include phosphate buffered saline (PBS), potassium phosphate, sodium phosphate and/or sodium citrate. Other biological buffers can include PIPES, MOPS etc.

A suitable aqueous citrate buffer may be a sodium citrate buffer or a potassium citrate buffer, for example a 50 mM sodium citrate buffer. A suitable phosphate buffer may be a sodium phosphate buffer, for example a 25 mM sodium phosphate buffer.

Suitable pH values for the composition include any generally acceptable pH values for administration in vivo, such as for example pH 5.0 to pH 9.0, suitably from pH 6.7 to pH 7.4, or pH6.8, pH 6.9, pH 7.0, pH 7.2. The pH may be adjusted accordingly with a suitable acid or alkali, for example hydrochloric acid.

The compositions of the invention may be formulated in an aqueous suspension ready for use, or the compositions may be prepared as a lyophilised formulation. Lyophilised formulations of the invention may be supplied as separate dosage forms along with a suitable diluent, adjuvant or excipient provided also, e.g. a physiologically acceptable buffer. As described herein, such compositions may additionally comprise Factor VIII as a separate dosage form, or formulated with the colloidal particles as described herein. Typically, a vial of lyophilised Factor VIII (FVIII) and a separate vial of colloidal particle (PEGLip) solution, for reconstitution will be provided.

The colloidal particle may be stored as a suspension of 9% (w/v) total lipids in an aqueous citrate buffer, suitably the particles may be stored as a suspension of 7%, 6%, 5%, 4% (w/v) total lipids. Once the required concentration of exogenous Factor VIII (FVIII) is known for the patient, the bulk solution may be diluted if necessary with 50 mM sodium citrate solution to adjust the concentration of the colloidal particles so that when the Factor VIII (FVIII) is added the desired ratio of colloidal particles to Factor VIII (FVIII) molecules is obtained.

The Factor VIII may be entirely exogenous and formulated with the invention prior to injection, for example in the case of a severe haemophiliac with inhibitors, for which use it may be either derived from plasma concentrates or recombinantly produced. Alternatively, if the patient retains some ability to self-manufacture Factor VIII (for example mild or moderate haemophiliacs, or patients with acquired haemophilia), a lesser amount or no exogenous Factor VIII will be administered.

Without wishing to be bound by theory, there is a presumption that the excess colloidal particles present in the composition in an amount sufficient to allow free colloidal particles to reversibly bind other core blood factors (for example FVII and FIX in the case of haemophilia A) which with an amount of particle-associated Factor VIII (FVIII) may be captured and reversibly bound to the platelets following administration, to concentrate factors at the platelet and boost the extrinsic blood coagulation pathway.

Upon injection the colloidal particle will reversibly bind to the surface of blood platelets and fuse with the membrane of others. Where the colloidal particle particles are already bound with an exogenous Factor VIII, this will concentrate the Factor VIII at the surface of the platelet with some maybe phagocytosed into the platelets or associated with or within the TF-bearing pro-coagulant microparticles that are produced, protecting the protein from inhibitors and also the normal clearance mechanisms, e.g. LRP-1, conferring a longer half-life on the protein. In patients with moderate or mild haemophilia, colloidal particles will also capture any circulating Factor VIII, concentrating it at or within the platelet or within the arising TF-bearing pro-coagulant microparticles. Colloidal particles that are not associated with Factor VIII on injection will begin to capture and concentrate FVII as well as other endogenous blood factors (e.g. FIX) at the surface of the platelets and to associate these with any TF-bearing pro-coagulant microparticles produced; it is also feasible that particles with no attached factors will also bind and fuse to the surface of the platelets, both forming TF-bearing pro-coagulant microparticles and acting as opportunistic traps to capture and concentrate further factors, including the activated forms, FVIIa and FIXa, at the platelet reaction surface during the maelstrom of the clotting cascade.

Ordinarily, on injury to the endothelium, tissue factor converts FVII to FVIIa and combines with it. The TF-FVIIa complex then migrates towards and binds onto the surface of the activated platelets and starts to convert FX to FXa to cleave prothrombin to generate thrombin, a process which becomes optimised when FXa complexes with FVIIa (released from the activated platelets) to form the prothrombinase complex, which is also assembled on the exposed membrane surfaces of the activated platelets and TF-bearing, pro-coagulatory microparticles derived from them. The invention places FVII in close proximity to this reaction surface, which may have shattered into many TF-bearing pro-coagulatory microparticles. Thus, the conversion to FVIIa (which remains bound to colloidal particle, and thus the platelet and microparticles) and the formation of the TF-FVIIa complex occurs on the reaction surface of the platelets and their microparticles with two important and immediate effects:
1) The localised conversion of FX to FXa, which combines with FV (the ‘prothrombinase complex’), also on these platelet and TF-bearing procoagulant microparticle surfaces, to produce a highly localised thrombin burst, which will both initiate the production of fibrin catalyse the amplification phase; and simultaneously
2) The localised conversion of FIX to FIXa which can then associate with the FVIII, which have been co-localised via the colloidal particle, to form the tenase complex on the same membrane surfaces, thus feeding and optimising the amplification phase that has been catalysed by the thrombin from the now augmented initiation phase in (1), above. Alternatively, and additionally the colloidal particle membrane may form a substitute tenase complex, attracting and converting FX to FXa

Once the clotting cascade is initiated and fibrin is produced, platelets ordinarily coagulate to infill the fibrin mesh. The ability of the colloidal particle to bind and fuse with platelets has a final role to play here in reinforcing adherence of the platelets together in the mesh to stabilise the clot.

The invention may act in multiple ways to improve the conversion of FX to FXa in the presence of both a limited amount of FVIII and inhibitors to FVIII. Firstly by protecting, enhancing and maximising the potential of a limited amount of FVIII to be able to form the tenase complex with FIXa to catalyse the conversion of FX to FXa; secondly, by mimicking the action of FVIIIa and binding FIXa to provide a substitute tenase complex to catalyse the conversion of FX to FXa; thirdly by upregulating the extrinsic pathway both through the production of TF-bearing pro-coagulant microparticles and by concentrating FVII/FVIIa to stimulate the conversion of FX to FXa through the extrinsic tenase complex; and finally, enhancing, via the upregulated extrinsic pathway, the conversion of FIX to FIXa to feed the formation of the intrinsic tenase complex. Through these actions the invention has multiple modes of action, by protecting, preserving and maximising the activity of FVIII in the tenase complex of the intrinsic pathway, while mimicking the functionality of FVIIIa in the tenase complex and also simultaneously bypassing that pathway through up-regulation of the extrinsic pathway.

The colloidal particle has a dual action, both as a bypassing agent to enhance FX to FXa conversion via the extrinsic pathway, as well as amplifying the intrinsic pathway, through both the protection of FVIII and by concentrating FIX/FIXa accelerating the formation of the tenase complex or forming a FVIII-independent tenase complex with FIXa. Together the enhanced initiation (extrinsic) and amplification (intrinsic) phases enable both the more rapid onset of clotting and the faster generation of fibrin that can be bound into a firmer clot than would normally be possible with such a reduced amount of Factor VIII—especially in the presence of inhibitory antibodies—leading to the faster resolution of a bleed for the patient.

The invention thus relies on the ability of the colloidal particle, and in particular its specific formulation ratio of colloidal particle to Factor VIII, both to concentrate correct amounts of both endogenous and exogenous Factor VIII at the platelet surface and inside the platelets, as well as stimulating the production of TF-bearing pro-coagulant microparticles, so that both the TF-FVIIa-centric initiation phase and the amplification phase of the clotting cascade are optimised together with the synergistic effect of accelerating the onset of thrombin generation with a limited amount of Factor VIII in the presence of inhibitors to Factor VIII in the case of haemophilia A).

Since the injected exogenous Factor VIII is protected from degradation by both inhibitors and normal clearance mechanisms, and since a better quality clot is formed faster both through concentrating the factors at the platelet, and the accelerating effect of the TF-bearing pro-coagulant microparticles, the invention will be Factor VIII sparing over other methods of supplying Factor VIII, as found by production of ectopic FVIII in platelets via gene therapy. This benefit will manifest in smaller or less frequent injections for patients, increasing compliance with prescribed treatment and decreasing the likelihood of an accidental and possibly fatal bleed.

While the invention concentrates exogenous Factor VIII and endogenous factors (FVII/FVIIa and FIX/FIXa) at and within the platelets, unlike the successful attempts to product FVIII ectopically in platelets via gene therapy it does not require the long-development programmes, regulatory burden or the irreversible nature of a virally-mediated transgene therapy.

By concentrating and amplifying the effect of a limited amount of Factor VIII that is at a lower than normal level in the disease to be treated (for example FVIII in the case of haemophilia A (HA)), it is anticipated that the invention will be Factor VIII-sparing, enabling either lower doses to be administered and/or reducing the number of injections that are normally required to achieve haemostasis in haemophilia patients and in particular in inhibitor patients who cannot normally be administered Factor VIII as their inhibitory antibodies will destroy the protein and leave them unprotected.

Unlike approaches to create novel engineered Factor VIII molecules or mimetics of these molecules or their activated forms, the invention can be used with any current plasma-derived or recombinant Factor VIII, without the need to engineer foreign sequences into the molecule, for example recombinant human FVIII (rhFVIII). This reduces the danger of an immunomodulatory response arising to a novel, unrecognised protein.

Unlike both of these approaches, i.e. the production of FVIII in platelets or the use of mimetics, the invention has the novel and very necessary dual action of not only concentrating an exogenously applied component of the extrinsic, acceleratory pathway, but in also both concentrating endogenous factors and stimulating the production of TF-bearing pro-coagulant microparticles to amplify the intrinsic pathway to a rapid thrombin burst and the local generation of the other major component (FIXa) of the extrinsic pathway, which may continue to drive the common pathway to thrombin production as Factor VIII levels fall again.

Use of the invention is Factor VIII-sparing over free Factor VIII. This means more convenience for patients (smaller injections), better compliance (fewer missed prophylactic injections) and better healthcare economics (less cost of Factor VIII, fewer emergency infusions when haemophiliacs have not been compliant and had bleeds).

By enabling the use of standard plasma-derived or recombinantly produced forms of Factor VIII it is an ultimate objective that the invention will enable a cost-effective solution to enabling prophylactic treatment with FVIII in haemophilia A patients with inhibitors.

Use of the invention is sparing over the use of exogenous FVIIa as a bypass agent in haemophilia patients with inhibitors. The invention not only uses the patient's own FVII but also both concentrates this at the platelets and stimulates the production of TF-bearing pro-coagulant microparticles to maximise the effectiveness of FVII, thus avoiding the cost of exogenous FVIIa and any concerns of thrombotic reactions due to overdosing with the protein.

The composition may be administered by injection or infusion, preferably intravenous, subcutaneous, intradermal or intramuscular. Injection comprises the administration of a single dose of the composition. Infusion comprises the administration of a composition over an extended period of time.

The compositions of the invention may be for administration at least once per day, at least twice per day, about once per week, about twice per week, about once per two weeks, or about once per month. The composition may also be administered and/or re-dosed at intervals to allow the blood concentration of FVIII to be maintained at a consistent level, providing a sustained, constant and predictable therapeutic effect without the need to wait to re-dose until the concentration of FVIII in the blood of the patient reaches sub-therapeutic or therapeutically irrelevant levels. In traditional practice, subsequent doses of FVIII are not normally given to the subject while “healthy levels”, or therapeutically effective/relevant levels, of FVIII are still present in the bloodstream. Thus, the invention provides for a more consistent therapeutic level of FVIII in the bloodstream that is more ideally suited to prophylaxis.

Sub-therapeutic or therapeutically irrelevant levels of FVIII in the blood of a subject may be characterised as being when a patient is not able to maintain a whole blood clotting time of 20 minutes, or less, 15 minutes, or less, or 12 minutes or less.

The invention provides a composition wherein a patient is able to maintain a whole blood clotting time of no more than 20 minutes, no more than 15 minutes or not more than 12 minutes.

It has been surprisingly found that formulations of blood factors in association with colloidal particles (liposomes) derivatized with a biocompatible polymer can be successfully administered subcutaneously and achieve a therapeutically effective dose of blood factor to a subject suffering from haemophilia.

In the examples of the present invention, the PEG is incorporated into the colloidal particle during vesicle formation, before association with the blood factor. It is believed that specific amino acid sequences on the blood factor may bind non-covalently to carbamate functions of the PEG molecules on the outside of the liposomes.

The colloidal particle does not encapsulate the blood factor. The blood factor interacts non-covalently with the polymer chains on the external surface of the liposomes, and no chemical reaction is carried out to activate the polymer chains. The nature of the interaction between the blood factor and the liposome derivatized with a biocompatible hydrophilic polymer may be by any non-covalent mechanism, such as ionic interactions, hydrophobic interactions, hydrogen bonds and Van der Waals attractions (Arakawa, T. and Timasheff, S. N., Biochemistry 24: 6756-6762 (1985); Lee, J. C. and Lee, L. L. Y., J. Biol. Chem. 226: 625-631 (1981)). An example of such a polymer is polyethylene glycol (PEG).

A variety of known coupling reactions may be used for preparing vesicle forming lipids derivatized with hydrophilic polymers. For example, a polymer (such as PEG) may be derivatized to a lipid such as phosphatidylethanolamine (PE) through a cyanuric chloride group. Alternatively, a capped PEG may be activated with a carbonyl diimidazole coupling reagent, to form an activated imidazole compound. A carbamate-linked compound may be prepared by reacting the terminal hydroxyl of MPEG (methoxyPEG) with p-nitrophenyl chloroformate to yield a p-nitrophenyl carbonate. This product is then reacted with 1-amino-2,3-propanediol to yield the intermediate carbamate. The hydroxyl groups of the diol are acylated to yield the final product. A similar synthesis, using glycerol in place of 1-amino-2,3-propanediol, can be used to produce a carbonate-linked product, as described in WO 01/05873. Other reactions are well known and are described, e.g. in U.S. Pat. No. 5,013,556.

Colloidal particles (liposomes) can be classified according to various parameters. For example, when the size and number of lamellae (structural parameters) are used as the parameters then three major types of liposomes can be described: Multilamellar vesicles (MLV), small unilamellar vesicles (SUV) and large unilamellar vesicles (LW).

MLV are the species which form spontaneously on hydration of dried phospholipids above their gel to liquid crystalline phase transition temperature (Tm). The size of the MLVs is heterogeneous and their structure resembles an onion skin of alternating, concentric aqueous and lipid layers.

SUV are formed from MLV by sonication or other methods such as extrusion, high pressure homogenisation or high shear mixing and are single layered. They are the smallest species with a high surface-to-volume ratio and hence have the lowest capture volume of aqueous space to weight of lipid.

The third type of liposome LUV has a large aqueous compartment and a single (unilamellar) or only a few (oligolamellar) lipid layers. Further details are disclosed in D. Lichtenberg and Y. Barenholz, in “Liposomes: Preparation, Characterization, and Preservation, in Methods of Biochemical Analysis”, Vol. 33, pp. 337-462 (1988).

As used herein the term “loading” means any kind of interaction of the biopolymeric substances to be loaded, for example, an interaction such as encapsulation, adhesion (to the inner or outer wall of the vesicle) or embedding in the wall with or without extrusion of the biopolymeric substances.

As used herein and indicated above, the term “liposome” refers to colloidal particles and is intended to include all spheres or vesicles of any amphipathic compounds which may spontaneously or non-spontaneously vesiculate, for example phospholipids where at least one acyl group replaced by a complex phosphoric acid ester. The liposomes may be present in any physical state from the glassy state to liquid crystal. Most triacylglycerides are suitable and the most common phospholipids suitable for use in the present invention are the lecithins (also referred to as phosphatidylcholines (PC)), which are mixtures of the diglycerides of stearic, palmitic, and oleic acids linked to the choline ester of phosphoric acid. The lecithins are found in all animals and plants such as eggs, soybeans, and animal tissues (brain, heart, and the like) and can also be produced synthetically. The source of the phospholipid or its method of synthesis are not critical, any naturally occurring or synthetic phosphatide can be used.

Examples of specific phosphatides are L-a-(distearoyl) lecithin, L-a-(dipalmitoyl) lecithin, L-a-phosphatide acid, L-a-(dilauroyl)-phosphatidic acid, L-a(dimyristoyl) phosphatidic acid, L-a(dioleoyl)phosphatidic acid, DL-a (di-palmitoyl) phosphatidic acid, L-a(distearoyl) phosphatidic acid, and the various types of L-a-phosphatidylcholines prepared from brain, liver, egg yolk, heart, soybean and the like, or synthetically, and salts thereof. Other suitable modifications include the controlled peroxidation of the fatty acyl residue cross-linkers in the phosphatidylcholines (PC) and the zwitterionic amphipathates which form micelles by themselves or when mixed with the PCs such as alkyl analogues of PC.

The phospholipids can vary in purity and can also be hydrogenated either fully or partially. Hydrogenation reduces the level of unwanted peroxidation, and modifies and controls the gel to liquid/crystalline phase transition temperature (Tm) which effects packing and leakage.

The liposomes can be “tailored” to the requirements of any specific reservoir including various biological fluids, maintains their stability without aggregation or chromatographic separation, and remains well dispersed and suspended in the injected fluid. The fluidity in situ changes due to the composition, temperature, salinity, bivalent ions and presence of proteins. The liposome can be used with or without any other solvent or surfactant.

Generally suitable lipids may have an acyl chain composition which is characteristic, at least with respect to transition temperature (Tm) of the acyl chain components in egg or soybean PC, i.e., one chain saturated and one unsaturated or both being unsaturated. However, the possibility of using two saturated chains is not excluded.

The liposomes may contain other lipid components, as long as these do not induce instability and/or aggregation and/or chromatographic separation. This can be determined by routine experimentation.

The PEGylated phospholipid may be physically attached to the surface of the colloidal particle or inserted into the membrane of the colloidal particle. The polymer may therefore be covalently bound to the colloidal particle.

A variety of methods for producing the modified colloidal particle which are unilamellar or multilamellar are known and available (see Lichtenberg and Barenholz, (1988)):
1. A thin film of the phospholipid is hydrated with an aqueous medium followed by mechanical shaking and/or ultrasonic irradiation and/or extrusion through a suitable filter;
2. Dissolution of the phospholipid in a suitable organic solvent, mixing with an aqueous medium followed by removal of the solvent;
3. Use of gas above its critical point (i.e., freons and other gases such as CO₂ or mixtures of CO₂ and other gaseous hydrocarbons) or
4. Preparing lipid detergent mixed micelles then lowering the concentration of the detergents to a level below its critical concentration at which liposomes are formed.

In general, such methods produce colloidal particles with heterogeneous sizes from about 0.02 to 10 μm or greater. Since colloidal particles which are relatively small and well defined in size are preferred for use in the present invention, a second processing step defined as “colloidal particle down-sizing” can be used for reducing the size and size heterogeneity of colloidal particle suspensions.

The colloidal particle suspension may be sized to achieve a selective size distribution of vesicles in a size range less than about 5 μm, for example <0.4 μm. In one embodiment of the invention, the colloidal particles have an average particle size diameter of from about 0.03 to 0.4 or 0.05 to 0.15 microns (μm), suitably around 0.1 microns (μm).

Colloidal particles in this range can readily be sterilized by filtration through a suitable filter. Smaller vesicles also show less of a tendency to aggregate on storage, thus reducing potentially serious blockage or plugging problems when the liposome is injected intravenously or subcutaneously. Finally, liposomes which have been sized down to the submicron range show more uniform distribution.

Several techniques are available for reducing the sizes and size heterogeneity of colloidal particle s, in a manner suitable for the present invention. Ultrasonic irradiation of a colloidal particle suspension either by standard bath or probe sonication produces a progressive size reduction down to small unilamellar vesicles (SUVs) between 0.02 and 0.08 μm in size.

Homogenization is another method which relies on shearing energy to fragment large colloidal particles into smaller ones. In a typical homogenization procedure, the colloidal particle suspension is recirculated through a standard emulsion homogenizer until selected liposome sizes, typically between about 0.1 and 0.5 μm are observed. In both methods, the particle size distribution can be monitored by conventional laser-beam particle size determination.

Extrusion of colloidal particles through a small-pore polycarbonate filter or equivalent membrane is also an effective method for reducing colloidal particle sizes down to a relatively well-defined size distribution whose average is in the range between about 0.02 and 5 μm, depending on the pore size of the membrane.

Typically, the suspension is cycled through one or two stacked membranes several times until the desired colloidal particle size distribution is achieved. The colloidal particle may be extruded through successively smaller pore membranes to achieve a gradual reduction in liposome size.

Centrifugation and molecular sieve chromatography are other methods which are available for producing a liposome suspension with particle sizes below a selected threshold less than 1 μm.

These two respective methods involve preferential removal of large liposomes, rather than conversion of large particles to smaller ones. Colloidal particle yields are correspondingly reduced.

The size-processed colloidal particle suspension may be readily sterilized by passage through a sterilizing membrane having a particle discrimination size of about 0.4 μm, such as a conventional 0.45 μm depth membrane filter. The liposomes are stable in lyophilized form and can be reconstituted shortly before use by taking up in water.

Suitable lipids for forming colloidal particle s are described above. Suitable examples include but are not limited to phospholipids such as dimirystoylphosphatidylcholine (DMPC) and/or dimirystoyl-phosphatidylglycerol (DMPG), egg and soybean derived phospholipids as obtained after partial or complete purification, directly or followed by partial or complete hydrogenation.

The following four methods are described in WO 95/04524 and are generally suitable for the preparation of the colloidal particles (liposomes) used in accordance with the present invention.

### Method A

a) mixing amphipathic substances, such as lipids suitable for forming vesicles in water-immiscible organic solvents
b) removing of the solvent in presence of a solid support, alternatively, dried amphipathic substances or mixtures thereof can be used in any form (powder, granular, etc.) directly,
c) taking up the product of step b) into a solution of the biopolymeric substances in a physiologically compatible solution
d) adding an organic solvent having solubilizing or dispersing properties, as well as
e) drying the fraction obtained in step d) under conditions retaining the function of the biopolymeric substances.

According to step a) of Method A amphipathic substances suitable for forming vesicles as mentioned above are mixed in a water-immiscible organic solvent. The water-immiscible organic solvent may be a polar-protic solvent such as fluorinated hydrocarbons, chlorinated hydrocarbons and the like.

In step b) of the method of the invention the solvent is removed in presence of a solid support. The solid support may be an inert organic or inorganic material having a bead-like structure. The material of the inorganic support material may be glass and the organic material can be Teflon™ or other similar polymers.

The step c) of Method A of the invention is for taking up the product of step b) into a solution of the substances to be encapsulated in a physiologically compatible solution.

The physiological compatible solution may be equivalent to a sodium chloride solution up to about 1.5 by weight. It is also possible to use other salts as long as they are physiologically compatible e.g. as a cryoprotectant e.g., sugars and/or amino acids. For example, lactose, sucrose or trehalose may be used as a cryoprotectant.

Optionally, between step a) and b) a step of virus inactivation, sterilizing, depyrogenating, filtering the fraction or the like of step a) can be provided. This might be advantageous in order to have a pharmaceutically acceptable solution at an early stage of the preparation.

The step d) of the Method A is adding an organic solvent having solubilizing or dispersing properties.

The organic solvent may be an organic polar-protic solvent miscible with water. Lower aliphatic alcohols having 1 to 5 carbon atoms in the alkyl chain can also be used, such as tertiary butanol (tert-butanol). The amount of organic polar-protic solvent miscible with water is strongly dependent on its interference with the substance to be loaded to the liposomes. For example, if a protein is to be loaded the upper limit is set by the amount of solvent by which the activity of the protein becomes affected. This may strongly vary with the nature of the substance to be loaded. For example, if the blood clotting factor comprises Factor IX then the amount of about of tert-butanol is around 30%, whereas, for Factor VIII an amount of less than 10% of tert-butanol is suitable (Factor VIII is much more sensitive to the impact of tert-butanol). The percentage of tert-butanol in these examples is based on percent by volume calculated for final concentration.

Optionally, subsequent to step d), virus inactivation sterilizing and/or portioning of the fraction yielded after step d) can be carried out.

The step e) of the present invention is drying the fraction obtained in step d) under conditions retaining the function of the substance to be loaded. One method for drying the mixture is lyophilization. The lyophilization may be carried out in presence of a cryoprotectant, for example, lactose or other saccharides or amino acids. Alternatively, evaporation or spray-drying can be used.

The dried residue can then be taken up in an aqueous medium prior to use. After taking up of the solid it forms a dispersion of the respective liposomes. The aqueous medium may contain a saline solution and the dispersion formed can optionally be passed through a suitable filter in order to down size the liposomes if necessary. Suitably, the liposomes may have a size of 0.02 to 5 μm, for example in the range of <0.4 μm.

The liposomes obtainable by the Method A show high loading of the blood factors.

The compositions of the invention can also be an intermediate product obtainable by isolation of either fraction of step c) or d) of the method A. Accordingly, the formulation of the invention also comprises an aqueous dispersion obtainable after taking up the product of step e) of method A in water in form of a dispersion (liposomes in aqueous medium).

Alternatively, the pharmaceutical compositions of the invention are also obtainable by the following methods which are referred to as Methods B, C, D and E.

### Method B

This method comprises also the steps a), b) and c) of the Method A. However, step d) and e) of Method A are omitted.

### Method C

In Method C step d) of method A is replaced by a freeze and thaw cycle which has to be repeated at least two times. This step is well-known in prior art to produce liposomes.

### Method D

Method D excludes the use of any osmotic component. In method D the steps of preparation of vesicles, admixing and substantially salt free solution of the substances to be loaded and co-drying of the fractions thus obtained is involved.

### Method E

Method E is simpler than methods A-D described above. It requires dissolving the compounds used for liposome preparation (lipids antioxidants, etc.) in a polar-protic water miscible solvent such as tert.-butanol. This solution is then mixed with an aqueous solution or dispersion containing the blood factor. The mixing is performed at the optimum volume ratio required to maintain the biological and pharmacological activity of the agent.

The mixture is then lyophilized in the presence or absence of cryoprotectant. Rehydration is required before the use of the liposomal formulation. These liposomes are multilamellar, their downsizing can be achieved by one of the methods described in WO 95/04524.

The composition for use in the treatment of haemophilia A in a subject of the first aspect of the invention may be used for a subject that has received less than 50 exposure days of a Factor VIII (FVIII) therapy. For example, the subject may never have received a Factor VIII (FVIII) therapy, such a patient is known as a previously untreated patient (PUP). For example, the subject may have received a Factor VIII (FVIII) therapy on alternate days for a total of 98 days therefore having received 49 exposure days of Factor VIII (FVIII) therapy.

A previously untreated patient (PUP) is defined as a subject who has never been treated with a Factor VIII (FVIII) therapy. PUPs are at the highest risk of developing a neutralising antibody against exogenous FVIII. Such a previously untreated patient is also known as an untreated patient or a patient that is naïve to a Factor VIII (FVIII) therapy.

A minimally treated patient (MTP) is defined as a subject who has received less than 50 exposure days of a Factor VIII (FVIII) therapy. MTPs are also at a risk of developing a neutralising antibody against exogenous FVIII.

A previously treated patients (PTP) is defined as a subject who has received greater than or equal to 50 exposure days of a Factor VIII (FVIII) therapy. A PTP is less likely to develop a neutralising antibody against exogenous FVIII.

The composition for use in the treatment of haemophilia A in a subject of the first aspect of the invention may be used for a paediatric subject. A paediatric patient is defined in the European Union (EU) as that part of the population aged between birth and 18 years. The paediatric population encompasses several subsets. The applied age classification of paediatric patients is:
pre-term and term neonates from 0 to 27 days;
infants (or toddlers) from 1 month to 23 months;
children from 2 years to 11 years; and
adolescents from 12 to less than 18 years.
   (see: http://eceuro a.eu/heath/sites/heathfies/fiIes/eudraex/voI-1/2014_c338_01/2014_c338_01_en.pdf)

The haemophilia A may be congenital haemophilia A (cHA) or acquired haemophilia A (aHA). Congenital haemophilia is an inherited bleeding disorder characterized by an absent or reduced level of clotting Factor VIII. Acquired haemophilia is an autoimmune condition in which there is sudden production of autoantibody inhibitors in an individual without any personal or family history of bleeding. The body produces autoantibodies against Factor VIII in haemophilia A.

A PUP or MTP may also have less than 5 Bethesda units of FVIII inhibitor (antibody) activity, i.e. the patient may have tested positive for the presence of inhibitors (antibodies) to FVIII. In keeping with the present invention, the patient may therefore be tested for the presence of FVIII inhibitors (antibodies to FVIII). The antibody inhibitors in a patient's blood are quantified using the Bethesda method and it is common parlance to use ‘Bethesda units’ when talking about the level of inhibitors (antibodies) in a patient. A value of greater than 5 Bethesda units of FVIII inhibitor is considered a high titre of inhibitor (see: https://www.ema.europa.eu/en/documents/scientific-guideline/guideline-clinical-investigation-recombinant-human-plasma-derived-factor-viii-products-revision-2_en.pdf).

The composition for use in the treatment of haemophilia A in a subject of the first aspect of the invention may also be used in the treatment of a subject that has less than 5 Bethesda units of FVIII inhibitor (antibody) activity.

A Bethesda unit (BU) is a measure of blood coagulation inhibitor activity. According to Practical Haemostasis, “1 Bethesda Unit (Bu) is defined as the amount of inhibitor in a plasma sample which will neutralise 50% of 1 unit of Factor VIII:C in normal plasma after 2 hr incubation at 37° C.” (Schumacher, Harold Robert (2000). Handbook of Hematologic Pathology. Informa Health Care, p. 583). 1 unit of Factor VIII is equal to 1 IU/mL Factor VIII.

The Bethesda assay is based on measuring residual FVIII activity remaining after dilutions of test plasma. The assay requires a comparison between a test mixture of test plasma and normal plasma and a control mixture of normal plasma and buffer incubated for 2 hours at 37° C. The percent residual activity in the test mixture is converted to Bethesda units (BU).

A Bethesda assay is performed as follows:
1. Doubling dilutions of test (patient) plasma [usually 1/2-1/1024] are made in Imadazole buffered saline and incubated with an equal volume of a normal plasma pool at 37° C. In the Nijmegen modification, the dilutions of patient plasma are made in Factor VIII deficient plasma.
2. A control mixture consisting of an equal volume of normal plasma mixed with Imadazole buffered saline (or in the case of the Nijmegen modification immunodepleted factor VIII deficient plasma) is prepared. The normal plasma pool will contain ˜100% [100 IU/dL] Factor VIII. This mixture actually has a starting concentration of 50% [50 IU/dL] Factor VIII (because you have performed a 50:50 dilution with buffer) but this does not matter because the same source and volume is added to all incubation mixtures. The use of the control compensates for the deterioration in Factors VIII and V during the incubation period.
3. At the end of the incubation period the residual factor VIII is assayed using a standard 1-stage APTT based assay using the incubated control as the 100% [100 IU/dL] standard.
4. The inhibitor concentration is calculated from a graph of residual factor VIII activity versus inhibitor units. The dilution of test plasma that gives a residual factor VIII nearest to 50% but within the range 30-60% is chosen for calculation of the inhibitor. It is also possible to calculate the inhibitor titre for each dilution and take the average. Any residual factor VIII <25% [25 IU/dL] or >75% [75 IU/dL] should NOT be used for the calculation of inhibitor level.
5. If the residual factor VIII activity is between 80-100% [80-100 IU/dL] the sample does not contain an inhibitor.
6. Derive the inhibitor titre from the graph and multiply by the dilution to give the final titre. A positive control plasma of known inhibitor titre should be included.

Levels of activity in the blood coagulation cascade may be measured by any suitable assay, for example the Whole Blood Clotting Time (WBCT) test, the Activated Partial Thromboplastin Time (APTT) or ROTEM. In the One stage and Two stage/Chromogenic assays, the blood samples have to be prepared by centrifugation to remove cellular fragments, mostly because the assay method involves spectrophotometry so the sample needs to be clear. The global clotting assays below assess the time course of the physical formation of a clot and are thus closer to ‘real life’ as all the components that contribute to a clot, e.g. the platelets, are included.

The Whole Blood Clotting Time (WBCT) test measures the time taken for whole blood to form a clot in an external environment, usually a glass tube or dish. WBCT can be assessed with 2 ml of whole blood taken immediately after collection and divided into two glass tubes. These two tubes are then placed into a 37° C. water bath and checked approximately every 20-30 seconds by gently tilting. A clot is determined when the tube can be inverted horizontally and there is no run-off of plasma and a solid clot is retained.

The Activated Partial Thromboplastin Time (APTT) test measures a parameter of part of the blood clotting pathway. It is abnormally elevated in haemophilia and by intravenous heparin therapy. The APTT requires a few millilitres of blood from a vein. The APTT time is a measure of one part of the clotting system known as the “intrinsic pathway”. The APTT value is the time in seconds for a specific clotting process to occur in the laboratory test. This result is always compared to a “control” sample of normal blood. If the test sample takes longer than the control sample, it indicates decreased clotting function in the intrinsic pathway. General medical therapy usually aims for a range of APTT of the order of 45 to 70 seconds, but the value may also be expressed as a ratio of test to normal, for example 1.5 times normal. A high APTT in the absence of heparin treatment can be due to haemophilia, which may require further testing.

ROTEM (rotational thromboelastometry) uses a ROTEM Delta 2.7.2 system to assess the coagulability of the blood samples via the NATEM assay (activated by re-calcification only). For the measurement 20 uL of CaCl₂) and 340 uL of citrated whole blood sample is placed in the apparatus. The assay is performed within 15 minutes of taking the fresh blood sample. The assay delivers a panoply of statistics during the formation of the clot, including the Clotting Time (CT—the time for the blood to start clotting), the Clot Formation Time (CFT—the time to maximum clot firmness) among others.

The invention provides a composition which enables the subject to maintain a whole blood clotting time of less than 20 minutes, less than 15 minutes, or suitably, less than 12 minutes.

The following describes the Chromogenic assay (sometimes called the “Two-stage Assay”) for assessing FVIII concentration.

FVIII plasma activity can be determined using a Chromogenix Coamatic Factor VIII chromogenic assay (Diapharma, K822585) with modifications to the supplied method as follows:
i. The inclusion of an amount of naïve plasma in the FVIII standard preparations to achieve comparability with plasma sample dilutions,
ii. The use of FVIII standards specific to each test article (Nuwiq™ or Factane™)
iii. The inclusion of additional FVIII activity values within two standard curve ranges.

### Preparation of Nuwiq™ and Factane™ FVIII Standard Stock Solutions:

A vial of each test article can be reconstituted to 100 IU/ml with purified water, stored frozen in small aliquots at −70° C. and an aliquot thawed at 37° C. on the day of the assay. The stock solution appropriate to the study test article is used for the analysis of the corresponding plasma samples.

The outline assay method was as follows:
1. A vial of Technoclone Factor VIII-deficient plasma (native; Diapharma 5154007) freshly reconstituted in 1 ml purified water,
2. FVIII standard working stock solution (1 IU/mL) freshly prepared by the addition of 0.010 mL of appropriate FVIII standard stock solution (100 IU/ml) to 0.990 mL FVIII-deficient plasma,
3. Coamatic kit Factor reagent, S-2765+I-2581 substrate and buffer working solution prepared according to kit instructions and pre-warmed to 37° C.,
4. 20% acetic acid stop solution was prepared,
5. A standard curve prepared from FVIII working stock solution (1 IU/ml) using a FVIII range appropriate to the study samples (see Tables 1 and 2),
6. One aliquot of each test plasma sample thawed quickly at 37° C.
7. 25 μl thawed test plasma samples diluted with 2000 μl of buffer working solution.
8. 50 μl of diluted FVIII standards and diluted test plasma samples added to the wells of a 96-well plate according to a plate map and incubated for 4 minutes at 37° C.
9. 50 μl of Factor reagent added to each well and incubated for 2 minutes at 37° C. (High range standard curve) or 4 minutes at 37° C. (Low range standard curve).
10. 50 μl of S-2765+I-2581 substrate added to each well and incubated for 2 minutes at 37° C. (High range standard curve) or 10 minutes at 37° C. (Low range standard curve).
11. 50 μl of 20% acetic acid stop solution added to each well. The colour in the wells turned a shade of yellow and the optical density was measured by a microplate reader at absorbance 405 nm.
12. FVIII standard activity (IU/ml) plotted against absorbance at 405 nm using a best-fit linear curve.
13. Test plasma sample absorbance read against the standard curve and the FVIII activity reported in IU/ml.
14. The mean (and median where indicated in individual studies) FVIII activity result calculated from the 3 mouse test plasma samples at each time-point and the data subjected to pharmacokinetic analysis.

Further tests for assessing FVIII concentration include:

### Chromogenic FVIII Activity Assay

The Biophen FVIII:C Assay Kit Ref #221406 was used with plasma samples diluted 1:10 in assay buffer and run against both a Nuwiq™ and a human plasma reference standard curve. Each curve was generated by serial dilution of FVIII in canine FVIII deficient plasma, then 1:10 dilution in assay buffer. The standard range in both curves was 0.003-0.4 U/mL, with linear range being 0.13-1.00 U/mL. Assay was performed as per kit protocol.

### One-Stage Factor VIII Assay—Siemens BCS-XP System

Samples were measured against a canine FVIII reference curve, generated using normal canine pooled plasma diluted in Owren's Veronal Buffer containing 2.5% canine FVIII deficient plasma. The range of the curve is 5-200%. Plasma samples were diluted 1:10 in Owren's Veronal Buffer, mixed with FVIII deficient plasma, then Actin FS was added. After an incubation of 3 min, activation with CaCl2 was initiated and time to clot was measured at 405 nm.

In accordance with the second aspect described above, the method of treating haemophilia A in a subject comprises the step of administering a composition comprising a colloidal particle. The colloidal particle comprises (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI). The second amphipathic lipid comprises a phospholipid moiety derivatised with a biocompatible hydrophilic polymer. The subject has received less than 50 exposure days to a Factor VIII (FVIII) therapy.

The biocompatible hydrophilic polymer may be selected from the group consisting of polyalkylethers, polylactic acids and polyglycolic acids, preferably, the biocompatible hydrophilic polymer is polyethylene glycol (PEG). The polyethylene glycol may have a molecular weight of between about 500 to about 5000 Daltons, preferably about 2000 Daltons or 5000 Daltons.

The phosphatidyl choline (PC) may be 1-palmitoyl-2-oleoyl-sn-glycero-3-phosphocholine (POPC).

The second amphipathic lipid may be N-(Carbonyl-methoxypolyethyleneglycol)-1,2-distearoyl-sn-glycero-3-phosphoethanolamine (DSPE-PEG) such as N-(Carbonyl-methoxypolyethyleneglycol-2000)-1,2-distearoyl-sn-glycero-3-phosphoethanolamine (DSPE-PEG2000) or N-(Carbonyl-methoxypolyethyleneglycol-5000)-1,2-distearoyl-sn-glycero-3-phosphoethanolamine (DSPE-PEG5000).

The colloidal particle may further comprise (iii) a non-ionic surfactant.

The composition may further comprise a Factor VIII (FVIII) molecule. Alternatively, the method may comprise a further step of separately or subsequently administering a composition comprising a Factor VIII (FVIII) molecule.

The composition comprising the colloidal particle and the composition comprising Factor VIII may be administered as part of a treatment regimen. Suitably, the composition comprising Factor VIII may be administered to a patient and 15, 30, 45, 60, 90, 120, 15 to 120, 15 to 60, 15 to 30 minutes later the composition comprising the colloidal particle is administered to the patient. The composition comprising the colloidal particle and the composition comprising Factor VIII may be administered and/or re-dosed at intervals to allow the blood concentration of FVIII to be maintained at a consistent level, providing a sustained, constant and predictable therapeutic effect without the need to wait to re-dose until the concentration of FVIII in the blood of the patient reaches sub-therapeutic or therapeutically irrelevant levels, suitably every 2, 3, 4, 5, 6, 7, 14, 21 days, such as 2 to 21 days, 4 to 14 days, 4 to 7 days. For example, the composition comprising Factor VIII may be administered to a patient 15 minutes before the composition comprising the colloidal particle is administered to the patient, with the two steps of administration repeated every 4 to 5 days.

Alternatively, the composition comprising the colloidal particle and the Factor VIII may be administered and/or re-dosed at intervals to allow the blood concentration of FVIII to be maintained at a consistent level, providing a sustained, constant and predictable therapeutic effect without the need to wait to re-dose until the concentration of FVIII in the blood of the patient reaches sub-therapeutic or therapeutically irrelevant levels, suitably every 2, 3, 4, 5, 6, 7, 14, 21 days, such as 2 to 21 days, 4 to 14 days, 4 to 7 days.

Such a treatment regimen reduces the amount of FVIII required to treat a patient suffering from haemophilia A.

The haemophilia A may be congenital haemophilia A (cHA) or acquired haemophilia A (aHA).

The subject may be a paediatric patient. The subject may also not have received a FVIII therapy.

The invention also includes uses of a composition comprising a colloidal particle in the manufacture of a medicament for the treatment of haemophilia A in a subject wherein the subject has received less than 50 exposure days to a Factor VIII (FVIII) therapy.

In accordance with the third aspect described above, the kit comprises (i) a composition comprising a colloidal particle and (ii) a composition comprising a Factor VIII (FVIII) molecule for use in the treatment of haemophilia A in a subject. The subject has received less than 50 exposure days to a Factor VIII (FVIII) therapy. The colloidal particle is be composed (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI). The second amphipathic lipid comprises a phospholipid moiety derivatised with a biocompatible hydrophilic polymer.

Lyophilised formulations of the invention may be supplied as separate dosage forms along with a suitable diluent, adjuvant or excipient provided also, e.g. a physiologically acceptable buffer. The colloidal particle and/or Factor VIII (FVIII) of the kit may be provided as a lyophilised formulation.

Alternatively, the Factor VIII (FVIII) of the kit may be provided as a lyophilised formulation and colloidal particle may be provided as a solution for reconstitution of the Factor VIII (FVIII). As described herein, such compositions may additionally comprise Factor VIII as a separate dosage form, or formulated with the colloidal particles as described herein. The lyophilised form of FVIII may be provided in a 500 IU vial. The colloidal particle and/or Factor VIII (FVIII) of the kit may be provided in aqueous form ready for use.

The colloidal particle may further comprise a non-ionic surfactant.

The kit optionally comprises instructions for use also.

In accordance with the fourth aspect described above, the kit comprises (i) a composition comprising a colloidal particle and (ii) a composition comprising a Factor VIII (FVIII) molecule for separate, simultaneous or subsequent use in the treatment of haemophilia A in a subject. The subject has received less than 50 exposure days to a Factor VIII (FVIII) therapy. The colloidal particle is be composed (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI). The second amphipathic lipid comprises a phospholipid moiety derivatised with a biocompatible hydrophilic polymer.

Lyophilised formulations of the invention may be supplied as separate dosage forms along with a suitable diluent, adjuvant or excipient provided also, e.g. a physiologically acceptable buffer. The colloidal particle and/or Factor VIII (FVIII) of the kit may be provided as a lyophilised formulation.

Alternatively, the Factor VIII (FVIII) of the kit may be provided as a lyophilised formulation and colloidal particle may be provided as a solution for reconstitution of the Factor VIII (FVIII). As described herein, such compositions may additionally comprise Factor VIII as a separate dosage form, or formulated with the colloidal particles as described herein. The lyophilised form of FVIII may be provided in a 500 IU vial. The colloidal particle and/or Factor VIII (FVIII) of the kit may be provided in aqueous form ready for use.

The colloidal particle may further comprise a non-ionic surfactant.

The kit optionally comprises instructions for use also.

In accordance with the fifth aspect described above the dosage form of a pharmaceutical composition comprises a colloidal particle comprising (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI), wherein said second amphipathic lipid comprises a phospholipid moiety derivatised with a biocompatible hydrophilic polymer for use in the treatment of haemophilia A in a subject. The subject has received less than 50 exposure days to a Factor VIII (FVIII) therapy.

The colloidal particle may further comprise a non-ionic surfactant.

The dosage form may be a provided as suitable containers or vials containing the appropriate dose for a patient, for example as a 250 IU, 500 IU, 750 IU or 1000 IU vial. The dosage form may also be provided as a tablet or in liquid form. The dosage form may also be in lyophilised form.

A surprising technical effect demonstrated by the invention is achieved by masking the epitopes of FVIII that would ordinarily provoke an immune response and the subsequent production of anti-FVIII antibodies.

Without wishing to be bound by theory it is thought that these benefits derive from the non-covalent association of PEGLip to the A3 domain of FVIII, thus shielding epitopes in the light chain domains of FVIII from recognition by the body's immune system; and/or preventing the endocytosis of FVIII by dendritic cells. The introduction of additional PEG provides additional binding sites, potentially concentrating FVIII on the liposomes.

The additional PEG provides a larger hydration sphere which provides greater shielding to the associated FVIII from the normal clearance mechanisms giving an extended period of haemostatic control by increasing the circulating half-life of the FVIII. The greater shielding may also provide better shielding of the epitopes on FVIII from antibody inhibitors, facilitating the use of the product in patients with inhibitors (antibodies) to FVIII.

The binding of the liposome to the A3 domain region of FVIII leaves the C1 & C2 domains of FVIII free to bind VWF, with the additional protection this provides.

It is believed that the fusion of PEGylated liposomes bearing FVIII will fuse with or be ingested by platelets and may play a role in the activation of the latter, creating of tissue factor-bearing, procoagulant microparticles which may upregulate the extrinsic pathway and accelerate clot formation. Introducing additional PEG-bearing lipid or surfactants into the liposome may destabilise the platelet membrane and further hasten this activation.

This effect is more pronounced in recombinant FVIII molecules, which are typically not administered with VWF which would naturally protect these epitopes in wild-type FVIII.

In addition to protection of the epitopes, the association with PEGLip may also extend the half-life of FVIII by protecting FVIII from the normal proteolytic clearance mechanisms, extending the dosing interval and reducing the total exposure of the patient to FVIII over time.

A surprising observation is described as follows:

A clinical trial was devised to examine the use of PEGLip+FVIII in patients with inhibitors, where it was hoped that the combination would prevent existing antibodies from attacking FVIII. As well as patients with existing antibodies, some patients were included who had a history of developing antibodies when presented with FVIII but were not currently exhibiting antibodies.

Surprisingly is was found that these patients did not develop new antibodies when presented with the PEGLip+FVIII combination with a colloidal particle to FVIII molecule ratio of 15:1 to 16:1, implying that the PEGLip was able to shield the epitopes from dendritic/B-cells and/or prevent the endocytosis of FVIII.

An experiment was also conducted in haemophilia A dogs involved IV dosing with PEGLip+human FVIII as a control with a colloidal particle to FVIII molecule ratio of 15:1 to 16:1. Such experiments are usually difficult as the animal's immune system naturally reacts to the foreign (human) protein by producing antibodies. Surprisingly in this case the animal did not produce antibodies to the human protein when it was administered in the presence of PEGLip.

Accordingly, the use of PEGLip, in combination with FVIII, prevents the generation of antibodies
in subjects that have a history of producing antibodies and should have produced antibodies to the applied rFVIII.
in non-human subjects when presented with a foreign protein (rFVIII) in combination with PEGLip.

Therefore, if PEGLip+FVIII cannot provoke an antibody response in patients/subjects that are historically prone to exhibiting such a response, the use of PEGLip+FVIII in previously untreated subjects (almost exclusively paediatric subjects) that have never produced antibodies to FVIII should prevent the incidence of inhibitor patients.

The following samples are made and tested according to the invention:
1. A series of colloidal particles (PEGLip) comprising a higher ratios of DSPE-PEG to POPC, wherein the PEG is PEG-2000.
2. A series of colloidal particles (PEGLip) comprising a ratios of DSPE-PEG to POPC, wherein the PEG is PEG-5000.
3. Colloidal particles according to point 1 and point 2 further comprising polysorbate 80.
4. A series of colloidal particles (F-PEGLip) comprising higher ratios of DSPE-PEG to POPC and/or high molecular weight PEG.

In certain embodiments, the following formulations are provided:

### 15 to 16:1 Formulation

PEGLip particles composed of 1-palmitoyl-2-oleoyl-sn-glycero-3-phosphocholine (POPC) and N-(Carbonyl-methoxypolyethyleneglycol-2000)-1,2-distearoyl-sn-glycero-3-phosphoethanolamine (DSPE-PEG(2000)) in a 97:3 molar ratio in a 50 mM sodium citrate buffer in a 9% suspension formulated with FVIII (Nuwiq™, Octapharma AG) in a ratio of PEGLip particle to FVIII molecule of between 15 to 16:1.

### 7 to 8:1 Formulation

PEGLip particles composed of 1-palmitoyl-2-oleoyl-sn-glycero-3-phosphocholine (POPC) and N-(Carbonyl-methoxypolyethyleneglycol-2000)-1,2-distearoyl-sn-glycero-3-phosphoethanolamine (DSPE-PEG(2000)) in a 97:3 molar ratio in a 50 mM sodium citrate buffer formulated in a 9% suspension with FVIII (Nuwiq™, Octapharma AG) in a ratio of PEGLip particle to FVIII molecule of between 7 to 8:1.

In alternative embodiments, the following formulations are provided:

### 15 to 16:1 Formulation

PEGLip particles composed of 1-palmitoyl-2-oleoyl-sn-glycero-3-phosphocholine (POPC), N-(Carbonyl-methoxypolyethyleneglycol-2000)-1,2-distearoyl-sn-glycero-3-phosphoethanolamine (DSPE-PEG(2000)) in a 97:3 molar ratio and polysorbate 80 in a 9:1 w/w ratio (POPC+DSPE-PEG(2000):polysorbate 80) in a 50 mM sodium citrate buffer in a 9% suspension formulated with FVIII (Nuwiq™, Octapharma AG) in a ratio of PEGLip particle to FVIII molecule of between 15 to 16:1.

### 7 to 8:1 Formulation

PEGLip particles composed of 1-palmitoyl-2-oleoyl-sn-glycero-3-phosphocholine (POPC), N-(Carbonyl-methoxypolyethyleneglycol-2000)-1,2-distearoyl-sn-glycero-3-phosphoethanolamine (DSPE-PEG(2000)) in a 97:3 molar ratio and polysorbate 80 in a 9:1 w/w ratio (POPC+DSPE-PEG(2000):polysorbate 80) in a 50 mM sodium citrate buffer formulated in a 9% suspension with FVIII (Nuwiq™, Octapharma AG) in a ratio of PEGLip particle to FVIII molecule of between 7 to 8:1.

In one particular embodiment of the invention, there is provided a composition for use in the treatment of haemophilia A in a subject who has received less than 50 exposure days to a Factor VIII (FVIII) therapy as follows:
colloidal particles composed of a first amphipathic lipid comprising a phosphatidyl choline moiety and a second amphipathic lipid comprising a phospholipid moiety selected from the group consisting of a phosphatidyl ethanolamine (PE), a phosphatidyl serine (PS) and a phosphatidyl inositol (PI), in a 97:3 molar ratio (9:1 w/w), for example a 97:3 molar ratio of 1-palmitoyl-2-oleoyl-sn-glycero-3-phosphocholine (POPC) and N-(Carbonyl-methoxypolyethyleneglycol-2000)-1,2-distearoyl-sn-glycero-3-phosphoethanolamine (DSPE-PEG2000) or a weight-corrected ratio if an equivalent molar ratio of a heavier PEGylated lipid is used for example a w/w ratio of between 4:1 and 5:1 of 1-palmitoyl-2-oleoyl-sn-glycero-3-phosphocholine (POPC) and N-(Carbonyl-methoxypolyethyleneglycol-5000)-1,2-distearoyl-sn-glycero-3-phosphoethanolamine (DSPE-PEG5000).
a diluent, such as a buffer (suitably at a physiologically acceptable pH, e.g. pH 6.5 to 7.2), for example a citrate buffer, optionally at a concentration of 50 mM.

In an alternative embodiment, there is provided a composition for use in the treatment of haemophilia A in a subject who has received less than 50 exposure days to a Factor VIII (FVIII) therapy as follows:
colloidal particles composed of a first amphipathic lipid comprising a phosphatidyl choline moiety and a second amphipathic lipid comprising a phospholipid moiety selected from the group consisting of a phosphatidyl ethanolamine (PE), a phosphatidyl serine (PS) and a phosphatidyl inositol (PI), in a 97:3 molar ratio (9:1 w/w), for example a 97:3 molar ratio of 1-palmitoyl-2-oleoyl-sn-glycero-3-phosphocholine (POPC) and N-(Carbonyl-methoxypolyethyleneglycol-2000)-1,2-distearoyl-sn-glycero-3-phosphoethanolamine (DSPE-PEG2000) or a weight-corrected ratio if an equivalent molar ratio of a heavier PEGylated lipid is used for example a w/w ratio of between 4:1 and 5:1 of 1-palmitoyl-2-oleoyl-sn-glycero-3-phosphocholine (POPC) and N-(Carbonyl-methoxypolyethyleneglycol-5000)-1,2-distearoyl-sn-glycero-3-phosphoethanolamine (DSPE-PEG5000). The colloidal particle further comprising a non-ionic surfactant selected from the group consisting of a polyoxyethylene sorbitan, a polyhydroxyethylene stearate and a polyhydroxyethylene laurylether, for example polyoxyethylene (20) sorbitan monooleate
a diluent, such as a buffer (suitably at a physiologically acceptable pH, e.g. pH 6.5 to 7.2), for example a citrate buffer, optionally at a concentration of 50 mM.

Preferred features for the second and subsequent aspects of the invention are as for the first aspect mutatis mutandis.

The present invention will now be described with reference to the following examples which are present for the purposes of illustration only and should not be construed as being limitations on the invention. Reference is also made to the following drawings in which:

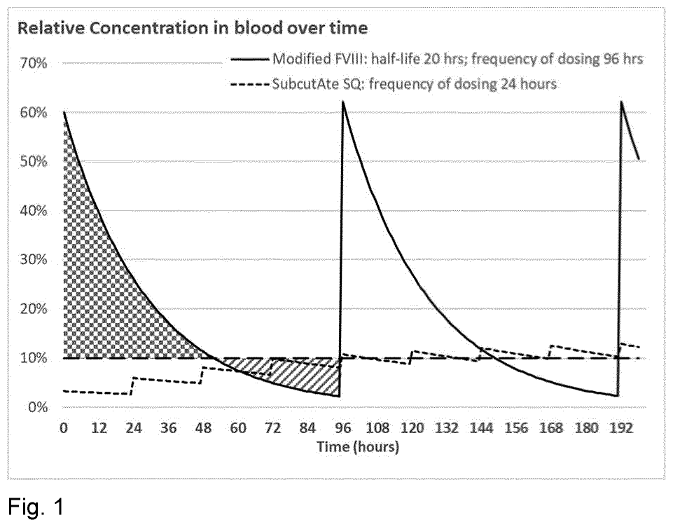

FIG. 1 illustrates intravenous dosing of extended half-life FVIII versus subcutaneously administered FVIII. Intravenous dosing of extended half-life FVIII either wastes FVIII (shaded area above 10% line) or leaves the patient with sub-therapeutic levels of FVII (shaded area below the line). Using a subcutaneously delivered depot can lead to a more economic and safer plateau of circulating FVIII. Both half-life and relative bioavailability are required to define a suitable SC dosing regimen (dose size and frequency)

### EXAMPLES

The Examples use the following formulation of PEGLip

| Ingredient (g per 100 g) | PEGLip (PLP-00) |
|---|---|
| POPC | 8.333 |
| mPEG-2000-DSPE | 0.926 |
| Polysorbate 80 | 0 |
| Sodium Citrate Dihydrate | 1.47 |
| Water | 89.271 |
| Total | 100 |
| pH | 6.5 to 7.2 |

### Example 1

### Studies of IV and SC-Administered PEGLip-FVIII in Haemophiliac Dogs

### Test Articles:

FVIII: Nuwiq™ (Octapharma, 500 IU vials); PEGLip: 9% PEGylated liposomes in 50 mM citrate buffer pH 6.7 (batch 19-740)
SubcutAte (3.5 ml 9% PEGLip per 500 IU vial of Nuwiq™ FVIII: 143 IU/ml PEGLip-FVIII)

### Method:

### Intravenous Administration of PEGLip-FVIII

PEGLip-FVIII dose of 50 IU/kg FVIII, 31.5 mg/kg PEGLip and 50 IU/kg FVIII (colloidal particle:FVIII molecule ratio of 16:1) was administered to a haemophilia A dog university of Queens at Kingston, Canada) by slow cephalic vein infusion. Venous blood samples were drawn from the cephalic vein at 30 mins, 2, 4, 8, 12, 24, 32, 48, and 56 hrs for whole blood clot times (WBCTs), Rotational thromboelastometry (ROTEM) analysis, FVIII:C activity levels, complete blood counts (CBC) and blood chemistry. Blood was also collected for Bethesda assay at 1- and 2-weeks post-treatment.

### Subcutaneous Administration

PEGLip-FVIII dose of 200 IU/kg FVIII, 126 mg/kg PEGLip and 200 IU/kg FVIII (colloidal particle:FVIII molecule ratio of 16:1) was administered to a haemophilia A dog University of Queens at Kingston, Canada) by subcutaneous (SQ) injection in the scruff of the neck. Venous blood samples were drawn from the cephalic vein at 1, 2, 4, 8, 12, 24, 32, 48 and 72 hrs for whole blood clot times (WBCTs), Rotational thromboelastometry (ROTEM) analysis, FVIII:C activity levels, complete blood counts (CBC) and blood chemistry. Blood was also collected for Bethesda assay at 1 and 2 weeks post-treatment.

### Results:

### Coagulation Correction

The single dose of intravenous PEGLip-FVIII substantially reduced clotting times from 30 minutes after administration and this effect hadn't returned to baseline after 56 hours. Subcutaneously administered FVIII would ordinarily be expected to be minimally bioavailable and ineffective at reducing clotting time. In contrast and surprisingly, the subcutaneous PEGLip-FVIII in this experiment was rapidly bioavailable and reduced clotting time from the first assessment time at 1 hour.

### Bioavailability and Half-Life

Bioavailability and half-life were analysed by looking at the decay in Clotting Time Reduction (CTR) over the first 48 hours after dosing, as assayed by Whole Blood Clotting Time (WBCT) or by ROTEM (Clotting Time (CT) plus Clot Formation Time (CFT).

Up to 23% bioavailability was calculated for the subcutaneous dose of PEGLip-FVIII. With the exception of the SC dose as measured by WBCT, the estimates of half-life far exceeded the literature estimates of Nuwiq™ half-life of 15.1-17.1 hours. See Table 1.

The combination of bioavailability and half-life enabled us to derive a potential subcutaneous dosing regimens comprising dose size and dosing interval, that would prevent FVIII levels from decreasing to a dangerous level (see FIG. 1).

TABLE 1

| | |
|---|---|
| Decay in CTR | Decay in CTR |
| via WBCT | via ROTEM |

| Administration | IV | SC | IV | SC |
|---|---|---|---|---|
| t_{1/2} (h) | 51.0 | 14.0 | 52.5 | 55.0 |
| AUC_{0-inf} | 613.5 | 219.9 | 5213.8 | 4832.6 |

| | | |
|---|---|---|
| Bioavailability | 9% | 23% |

### Inhibitor Generation

In usual practice with human FVIII injected IV, the haemophilic dogs in this colony will always develop anti-human FVIII antibodies of varying titres between 10 days and 4 weeks after FVIII dosing. However, in this study report the IV-dosed subject developed no inhibitors. This is surprising and highly unusual for an animal dosed with a human protein.

The SC-dosed animals did develop low levels of inhibitors of between 4 and 17 BU but this is lower than would typically be expected with intravenous dosing (typically 50-100 BU) and against a background of a large dose of 200 IU/kg of a protein from a different species being delivered in a single large volume to an immunogenic tissue. This may suggest that even the single high dose of PEGLip-FVIII administered SC was less immunogenic than might be expected with an equivalent IV dose of rhFVIII alone.

These surprising results suggest that, without compromising FVIII activity and without modifying the epitopes on the protein, the use of PEGLip as a coadministration agent with FVIII is able to prevent or reduce the generation of inhibitors in subjects where an immunogenic response would ordinarily be expected. This suggests that the co-administration of PEGLip with FVIII reduces the immunogenicity of the latter, making it a safer alternative to give to previously untreated patients, or minimally-treated patients who have not yet developed inhibitors

### Example 2

### SelectAte™ Clinical Study

Studies are underway to demonstrate that PEGLip-FVIII at a determined ratio of restores coagulation in inhibitor patients.

Inhibitor-prone HA patients did not generate inhibitors when injected with PEGLip:FVIII, while still showing coagulation correction

Four severe haemophiliac patients with a history of generating antibody inhibitors to FVIII presented to the trial. Due to their history, these patients are unable to receive replacement FVIII as a prophylactic therapy due to the risk of them generating inhibitors. In the trial three of these patients initially presented without inhibitors and one presented with a low titre (<5 BU). All patients were dosed with PEGLip-FVIII at 22 mg/kg PEGLip and 35 IU/kg recombinant humanised FVIII (a vesicle:FVIII ratio of 15:1 to 16:1). Over the first week of assessment, all patients experienced significant coagulation correction, enabling them to be dosing every 6 days on average (range 4-7 days) over the following 6 weeks. Despite re-dosing on a once weekly basis over 6 weeks, none of the three inhibitor-prone patients who presented without inhibitors generated inhibitors to the treatment. The one patient who presented with low titre inhibitors experienced a small, clinically insignificant rise during the first stage which actually reduced during the repeated dosing on the second phase. It is proposed that the inability of the PEGLip-FVIII to stimulate inhibitor generation in inhibitor-prone individuals will make the product exceptionally suitable for the treatment of previously untreated patients or minimally treated patients to prevent the generation of inhibitors in these vulnerable individuals.

TABLE 2

| Severe HA Patients with Inhibitors | Mean | Median |
|---|---|---|
| (N = 4) | (SD) | (Min; Max) |
| Frequency of PEGLip-FVIII injections | 6.0 | 6.5 |
| determined in Stage A (days) | (1.4) | (4.0; 7.0) |
| Frequency of PEGLip-FVIII injections | 5.1 | 5.2 |
| administered during Stage B (days) | (1.2) | (3.6; 6.3) |
| History of spontaneous bleeding episodes | 0.9 | 0.8 |
| during 24 weeks prior to enrollment | (0.4) | (0.5; 1.3) |
| (episodes/month) | | |
| Spontaneous bleeding episodes evaluated | 0.5 | 0.0 |
| during Stage B (episodes/month) - 3 of | (0.9) | (0.0; 0.9) |
| the 4 patients did not experience any | | |
| spontaneous bleeds | | |
| Anti-FVIII antibodies (Bethesda Units) - | 1.1 | 0.0 |
| Screening | (2.2) | (0.0; 4.4) |
| Anti-FVIII antibodies (Bethesda Units) - | 2.0 | 0.0 |
| End of Stage A | (4.0) | (0.0; 8.0) |
| Anti-FVIII antibodies (Bethesda Units) - | 1.7 | 0.0 |
| End of Stage B | (3.4) | (0.0; 6.8) |

### Example 3

### Clinical Study of PEGLiP+FVIII in Inhibitor-Prone and Inhibitor-Presenting Patients

A Phase 2 clinical study was undertaken to demonstrate that PEGLip-FVIII restores coagulation in inhibitor-prone and inhibitor-presenting patients, without stimulating the production of inhibitors in inhibitor-prone patients or causing an increase in inhibitor titre in inhibitor-presenting patients. The study built upon the findings described in Example 2.

Severe HA patients who either had inhibitors at screening, or whose clinical record showed them to be prone to develop inhibitors on exposure to FVIII did not increase their inhibitor titre or generate inhibitors, respectively, when injected with PEGLiP+FVIII (PLP-00+simoctocog alfa), while still showing coagulation correction and a reduction in bleeding frequency

Thirteen severe haemophiliac patients with a history of generating antibody inhibitors to FVIII presented to the trial. Due to their history, eight of these patients are unable to receive replacement FVIII as a prophylactic therapy due to the risk of them generating inhibitors. The other 5 patients presented at screening with active inhibitor titres (mean 2.4 Bethesda Units). All patients were dosed with PEGLip+FVIII (simoctoctog alfa) at 22 mg/kg PEGLip and 35 IU/kg recombinant humanised FVIII (a colloidal particle:FVIII ratio of 15:1 to 16:1). Over the first week of assessment, all patients experienced significant coagulation correction, enabling them to be dosing every 5.5 days on average (range 3.0-7.4 days) over the following 6 weeks, representing a significant extension in dosing interval (normal dosing interval for simoctoctog alfa is every other day or 2-3 times per week). Despite this lower frequency of dosing, bleeding events were reduced significantly in the patients, with average monthly bleed rates dropping from an average of 1 per month (12.3 per annum) to 0.3 per month (3.2 per annum).

Despite re-dosing on a once weekly basis over 6 weeks, none of the eight inhibitor-prone patients who presented without inhibitors generated inhibitors to the treatment. In addition, the five patients who presented with inhibitors did not experience a significant rise in inhibitor titre during the 6 weeks prophylactic stage of that trial. It is proposed that as well as a treatment for inhibitor-presenting and inhibitor-prone patients, the inability of the PEGLip-FVIII to stimulate inhibitor generation in inhibitor-prone individuals will additionally make the product exceptionally suitable for the treatment of previously untreated patients or minimally treated patients to prevent the generation of inhibitors in these vulnerable individuals.

TABLE 3

| | | Inhibitor | |
|---|---|---|---|
| | Inhibitor | patients | |
| | patients | Active | Total |
| | No active | inhibitors | inhibitor |
| | inhibitors | at screening | patients |
| n= | 8 | 5 | 13 |

Bethesda titres (units

| | | | |
|---|---|---|---|
| At screening | | | |
| Mean (SD) units | 0.0 (0) | 2.4 (1.3) | 0.9 (1.4) |
| Median [Min; Max] units | 0.0 [0; 0] | 2.0 | 0.0 |
| | | [1.3; 4.4] | [0.0; 4.4] |
| At end of stage A | | | |
| Mean (SD) units | 0.0 (0) | 2.5 (3.1) | 1.0 (2.2) |
| Median [Min; Max] units | 0.0 [0; 0] | 1.1 | 0.0 |
| | | [1.0; 8.0] | [0.0; 8.0] |
| At end of stage B | | | |
| Mean (SD) units | 0.0 (0) | 2.4 (2.5) | 0.9 (1.9) |
| Median [Min; Max] units | 0.0 [0; 0] | 1.3 | 0.0 |
| | | [0.9; 6.8] | [0.0; 6.8] |
| Change (Stage B-screening) | 0 | 0 | 0.0 |
| (p-value) | n/a | P = 0.975 | p > 0.999 |

Frequency of PEGLip-FVIII injections administered during Stage B (days)

| | |
|---|---|
| Mean (SD) | 5.5 (1.3) |
| Median [Min; Max] | 5.5 |
| | [3.0; 7.4] |

Bleeding Episodes per month

| | | | |
|---|---|---|---|
| 24 weeks prior to enrolment | | | |
| Mean (SD) | 0.9 (0.3) | 1.2(0.2) | 1.0 (0.3) |
| Median [Min; Max] | 1.0 | 1.3 | 1.0 |
| | [0.5; 1.3] | [0.8; 1.3] | [0.5; 1.3] |
| During Stage B | | | |
| Mean (SD) | 0.2 (0.7) | 0.3 (0.4) | 0.3 (0.6) |
| Median [Min; Max] | 0.0 | 0.0 | 0.0 |
| | [0.0; 1.9] | [0.0; 0.8] | [0.0; 1.9] |
| Mean (Stage B - Screening) | −0.7 | −0.9 | −0.8 |
| (p-value) | | | p < 0.005 |

### Conclusion

A ratio of between 15:1 to 16:1 of PEGLUP to FVIII both lowers the risk of bleeding events in patients with inhibitors to FVIII, as well as in patients who are prone to developing inhibitors to FVIII, without stimulating the production of further, significant amounts of inhibitors.

### Example 4

### Clinical Study of PEGLip Enhancement of FVIII in Non-Inhibitor Patients

A Phase 2 clinical study was undertaken to demonstrate that PEGLip injected after a prophylactic dose of FVIII allows the dosing interval of prophylactic doses of FVIII to be safely extended, while still preventing bleeding episodes, thus reducing the exposure of the patients to FVIII.

Fourteen severe haemophiliac patients without inhibitors. All patients were dosed intravenously with FVIII (simoctocog alfa) at 35 IU/kg, which was followed after 15 minutes by a single intravenous infusion of PEGLip at 22 mg/kg.

Over the first week of assessment, all patients experienced significant coagulation correction, enabling them to be dosing every 4.9 days on average (range 3.1-7.3 days) over the following 6 weeks, representing a significant extension in dosing interval (normal dosing interval for simoctocog alfa is every other day or every 2-3 times per week).

Despite this lower frequency of dosing, bleeding events were reduced significantly in the patients, with average monthly bleed rates dropping from an average of 1.10 per month (13.21 per annum) to 0.18 per month (2.21 per annum).

Over 6 weeks of prophylactic treatment, none of the patients developed inhibitors to FVIII.

It is proposed that the ability of PEGLip, when injected subsequently to a patient's normal standard of care FVIII to extend the dosing interval of that FVIII in this way can halve the number of exposures to FVIII for previously untreated/minimally treated patients, reducing the possibility of developing a immunogenic response.

TABLE 4

| | History | | |
|---|---|---|---|
| | (24 weeks prior | Stage B | |
| | to enrolment) | (6 weeks | Difference |
| n | 14 | 14 | 14 |
| Monthly Bleed Rate | | | |
| (events/month) | | | |
| Mean (SD) | 1.10 (0.64) | 0.18 (0.37) | −0.92 (0.63) |
| Median [Min, Max] | 1.08 | 0.00 | −0.96 |
| | [0.58; 1.58] | [0.00; 0.00] | |
| probability | | | p = 0.019 |
| Dosing interval during | | | |
| Stage B (days) | | | |
| Mean (SD) | 1.08 | 4.9 (1.4) | |
| | [0.25; 2.00] | | |
| Median [Min, Max] | | 0.00 | |
| | | [0.00; 1.09] | |

1. A composition comprising a colloidal particle comprising (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI),
wherein said second amphipathic lipid comprises a phospholipid moiety derivatised with a biocompatible hydrophilic polymer,
for use in the treatment of haemophilia A in a subject,
wherein the subject has received less than 50 exposure days to a Factor VIII (FVIII) therapy.

2. The composition for use of claim 1, wherein the biocompatible hydrophilic polymer is selected from the group consisting of polyalkylethers, polylactic acids and polyglycolic acids.

3. The composition for use of claim 1 or claim 2, wherein the biocompatible hydrophilic polymer is polyethylene glycol (PEG).

4. The composition for use of claim 3, wherein the polyethylene glycol has a molecular weight of between about 500 to about 5000 Daltons.

5. The composition for use of claim 4, wherein the polyethylene glycol has a molecular weight of about 2000 Daltons or about 5000 Daltons.

6. The composition for use of any one of claims 1 to 5, wherein the phospholipid is N-(Carbonyl-methoxypolyethyleneglycol)-1,2-distearoyl-sn-glycero-3-phosphoethanolamine (DSPE-PEG).

7. The composition for use of any one of claims 1 to 6, wherein the phospholipid is N-(Carbonyl-methoxypolyethyleneglycol-2000)-1,2-distearoyl-sn-glycero-3-phosphoethanolamine (DSPE-PEG2000) or N-(Carbonyl-methoxypolyethyleneglycol-5000)-1,2-distearoyl-sn-glycero-3-phosphoethanolamine (DSPE-PEG5000).

8. The composition for use of any one of claims 1 to 7, wherein the phosphatidyl choline (PC) is 1-palmitoyl-2-oleoyl-sn-glycero-3-phosphocholine (POPC).

9. The composition for use of any one of claims 1 to 8, wherein the composition comprises the first amphipathic lipid and the second amphipathic lipid in a molar ratio of from 90 to 99:10 to 1.

10. The composition for use of claim 9, wherein the composition comprises the first amphipathic lipid and the second amphipathic lipid in a molar ratio of 97:3.

11. The composition for use of any one of claims 1 to 10, wherein the colloidal particle further comprises (iii) a non-ionic surfactant selected from the group consisting of a polyoxyethylene sorbitan, a polyhydroxyethylene stearate and a polyhydroxyethylene laurylether.

12. The composition for use of claim 11, wherein the non-ionic surfactant is polyoxyethylene (20) sorbitan monooleate.

13. The composition for use of claim 11 or claim 12, wherein the colloidal comprises the first amphipathic lipid and the second amphipathic lipid to the non-ionic surfactant in a ratio of from 30:1 to 2:1 w/w.

14. The composition for use of claim 1, wherein the composition comprises the first amphipathic lipid to the second amphipathic lipid to the non-ionic surfactant in a ratio of from 10 to 40:1:0 to 4 w/w.

15. The composition for use of any one of claims 1 to 14, wherein the composition further comprises a Factor VIII (FVIII) molecule.

16. The composition for use of claim 15, wherein the composition comprises the colloidal particle and the Factor VIII (FVIII) molecule in a stoichiometric ratio of from 1 to 90:1.

17. The composition for use of claim 15 or claim 16, wherein the composition comprises the colloidal particle and the Factor VIII (FVIII) molecule in a stoichiometric ratio of 10 to 20:1 or 5 to 10:1.

18. The composition for use of any one of claims 1 to 17, wherein the haemophilia A is congenital haemophilia A (cHA).

19. The composition for use of any one of claims 1 to 17, wherein the haemophilia A is acquired haemophilia A (aHA).

20. The composition for use of any one of claims 1 to 19, wherein the composition further comprises a therapeutically active compound.

21. The composition for use of any one of claims 1 to 20, wherein the composition further comprises an excipient, diluent or adjuvant.

22. The composition for use of any one of claims 1 to 21, wherein the subject is a paediatric subject.

23. The composition for use of any one of claims 1 to 22, wherein the subject has not received a FVIII therapy.

24. A method of treating a haemophilia A in a subject comprising the step of:
administering a composition comprising a colloidal particle comprising (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI),
wherein said second amphipathic lipid comprises a phospholipid moiety derivatised with a biocompatible hydrophilic polymer, and
wherein the subject has received less than 50 exposure days to a Factor VIII (FVIII) therapy.

25. The method of claim 24, wherein the biocompatible hydrophilic polymer is selected from the group consisting of polyalkylethers, polylactic acids and polyglycolic acids.

26. The method of claim 24 or claim 25, wherein the biocompatible hydrophilic polymer is polyethylene glycol (PEG).

27. The method of claim 26, wherein the polyethylene glycol has a molecular weight of between about 500 to about 5000 Daltons.

28. The method of claim 27, wherein the polyethylene glycol has a molecular weight of about 2000 Daltons or about 5000 Daltons.

29. The method of any one of claims 24 to 28, wherein the phospholipid is N-(Carbonyl-methoxypolyethyleneglycol)-1,2-distearoyl-sn-glycero-3-phosphoethanolamine (DSPE-PEG).

30. The method of any one of claims 24 to 29, wherein the phospholipid is N-(Carbonyl-methoxypolyethyleneglycol-2000)-1,2-distearoyl-sn-glycero-3-phosphoethanolamine (DSPE-PEG2000) or N-(Carbonyl-methoxypolyethyleneglycol-5000)-1,2-distearoyl-sn-glycero-3-phosphoethanolamine (DSPE-PEG5000).

31. The method of any one of claims 24 to 30, wherein the phosphatidyl choline (PC) is 1-palmitoyl-2-oleoyl-sn-glycero-3-phosphocholine (POPC).

32. The method of any one of claims 24 to 31, wherein the colloidal particle further comprises (iii) a non-ionic surfactant.

33. The method of any one of claims 24 to 32, wherein the composition further comprises a Factor VIII (FVIII) molecule.

34. The method of any one of claims 24 to 32, wherein the method comprises a further step of separately or subsequently administering a composition comprising a Factor VIII (FVIII) molecule.

35. The method of any one of claims 24 to 34, wherein the haemophilia A is congenital haemophilia A (cHA).

36. The method of any one of claims 24 to 34, wherein the haemophilia A is acquired haemophilia A (aHA).

37. The method of any one of claims 24 to 36, wherein the subject is a paediatric subject.

38. The method of any one of claims 24 to 37, wherein the subject has not received a FVIII therapy.

39. A kit comprising (i) a composition comprising a colloidal particle and (ii) a composition comprising a Factor VIII (FVIII) molecule for use in the treatment of haemophilia A in a subject, wherein the subject has received less than 50 exposure days to a Factor VIII (FVIII) therapy,
wherein the colloidal particle comprises (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI),
wherein said second amphipathic lipid comprises a phospholipid moiety derivatised with a biocompatible hydrophilic polymer.

40. The kit of claim 39, wherein the colloidal particle further comprises (iii) a non-ionic surfactant.

41. A kit comprising (i) a composition comprising a colloidal particle and (ii) a composition comprising a Factor VIII (FVIII) molecule for separate, simultaneous or subsequent use in the treatment of haemophilia A in a subject, wherein the subject has received less than 50 exposure days to a Factor VIII (FVIII) therapy,
wherein the colloidal particle comprises (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI),
wherein said second amphipathic lipid comprises a phospholipid moiety derivatised with a biocompatible hydrophilic polymer.

42. The kit of claim 41, wherein the colloidal particle further comprises (iii) a non-ionic surfactant.

43. A dosage form of a pharmaceutical composition comprising a colloidal particle comprising (i) a first amphipathic lipid comprising a phosphatidylcholine (PC) moiety and (ii) a second amphipathic lipid comprising a phospholipid moiety selected from the group consisting of a phosphatidyl ethanolamine (PE), a phosphatidyl serine (PS) and a phosphatidyl inositol (PI),
wherein said second amphipathic lipid comprises a phospholipid moiety derivatised with a biocompatible hydrophilic polymer for use in the treatment of haemophilia A in a subject,
wherein the subject has received less than 50 exposure days to a Factor VIII (FVIII) therapy.

44. The dosage form of claim 43, wherein the colloidal particle further comprises (iii) a non-ionic surfactant.
